(19) United States
(12) Patent Application Publication
(10) Pub. No.: US 2011/0218451 A1
(43) Pub. Date: Sep. 8, 2011

(54) NASAL DEVICES, SYSTEMS AND METHODS

(76) Inventors: Danny Yu-Youh Lai, San Jose, CA (US); Arthur Ferdinand, San Jose, CA (US); Michael P. Wong, San Francisco, CA (US); Elliot Sather, San Francisco, CA (US); Rajiv Doshi, Los Altos, CA (US); Bryan Loomas, Los Gatos, CA (US); Toru Mino, Chicago, IL (US); Jonathan Patrick Summers, Redwood City, CA (US); Arthur G. Sandoval, San Francisco, CA (US); Jonathan Downing, San Francisco, CA (US); Jeffrey W. Servaites, San Francisco, CA (US); Ryan K. Pierce, San Francisco, CA (US); Motohide Hatanaka, Tokyo (JP)

(21) Appl. No.: 13/062,888

(22) PCT Filed: Sep. 15, 2009
(86) PCT No.: PCT/US2009/056948

§ 371 Date: May 17, 2011

Related U.S. Application Data
(60) Provisional application No. 61/097,187, filed on Sep. 15, 2008.
(60) Provisional application No. 61/103,545, filed on Oct. 7, 2008.
(60) Provisional application No. 61/105,292, filed on Oct. 14, 2008.

(51) Int. Cl. A61F 5/56; A61B 5/085

(52) U.S. Cl. 600/533; 128/848

(57) ABSTRACT

Described herein are passive nasal device having a resistance to exhalation that is greater than the resistance to inhalation. Also described are devices, methods and systems for sensing and measuring intranasal pressure when a subject is wearing a passive nasal respiratory device that is configured to inhibit exhalation more than inhalation. Also described are adapters for nasal devices and methods of using a nasal device adapter. Adapters may be used so that a passive nasal device may be applied indirectly in communication with a subject's nose; in some variations this may allow the passive nasal device to be re-used. Also described herein are nasal devices having a billowing airflow resistor that is configured to have a greater resistance to exhalation than to inhalation. The billowing airflow resistor typically includes a first layer that is adjacent to a second layer; the first layer is flexible and billows opens during inhalation so that the first layer remains separated from the second layer, but remains substantially parallel to the second layer. During exhalation, the first layer collapses back down against the second layer. Additional passive nasal devices, systems and methods of using them are also described.

### CROSS REFERENCE TO RELATED APPLICATIONS

This application claims the benefit of the following provisional patent applications: U.S. Provisional Patent Application Ser. No. 61/097,187, titled “NASAL DEVICE WITH INTRANASAL PRESSURE DETECTION,” filed Sep. 15, 2008; U.S. Provisional Patent Application Ser. No. 61/105,292, titled “NASAL DEVICES INCLUDING BILLOWING VALVES,” filed Oct. 14, 2008; and U.S. Provisional Patent Application Ser. No. 61/103,545, titled “NASAL DEVICES, SYSTEMS AND METHODS,” filed Oct. 7, 2008.

This provisional patent application may also be related to U.S. patent application Ser. No. 12/044,868, filed Mar. 7, 2008 (titled “RESPIRATORY SENSOR ADAPTERS FOR NASAL DEVICES”), claiming priority to U.S. Provisional Patent Application Ser. No. 60/905,850 (filed Mar. 7, 2007).

### INCORPORATION BY REFERENCE

All publications, including patents and patent applications, mentioned in this specification are herein incorporated by reference in their entirety as if each individual publication was specifically and individually indicated to be incorporated by reference.

### FIELD

Described are nasal devices and methods of using nasal devices to treat breathing disorders. In particular, the devices and methods described herein may be used to treat sleep disorders, such as snoring and sleep apnea. Among the inventions described herein are nasal respiratory devices (“nasal devices”) that may be placed in communication with the subject's nose and used to treat respiratory disorder including sleeping disorders such as snoring and sleep apnea. In particular, nasal respiratory disorders having one or more billowing airflow resistors are described. Also described herein are systems, methods and devices for measuring intranasal pressure, including nasal devices having a sensor on (or connection to a sensor from) the intranasal side of the device.

### BACKGROUND

Nasal respiratory devices may be worn to treat many medical conditions, such as sleep disordered breathing (including snoring, sleep apnea, etc.), Cheyne Stokes breathing, UARS, COPD, hypertension, asthma, GERD, heart failure, and other respiratory and sleep conditions. Examples of nasal respiratory devices that are of particular interest are nasal respiratory devices that are adapted to be removably secured in communication with a nasal cavity, and may include an opening configured to be placed in communication with the subject's nose that has a valve (or other airflow resistor) in communication with the opening, and a holdfast in communication with the opening that secures the device at least partially over and/or at least partially within the subject's nasal opening(s) so that the inhalation and exhalation can be regulated by the device, typically inhibiting nasal exhalation more than nasal inhalation. The holdfast is thus configured to removably secure the respiratory device at least partly within (and/or at least partly over and/or at least partly around) the nasal cavity. The airflow resistor (which may be a valve) is typically configured to provide greater resistance during exhalation than during inhalation.

Such nasal respiratory devices have been described for treatment of respiratory disorders such as sleep disordered breathing (e.g., apnea). For example, nasal devices and associated devices, systems and methods of use are described in: U.S. Pat. No. 7,506,649, titled “NASAL DEVICES” (filed Jun. 7, 2007); U.S. patent application Ser. No. 11/805,496 “NASAL RESPIRATORY DEVICES” (filed May 22, 2007); U.S. patent application Ser. No. 11/759,916 “LAYERED NASAL DEVICES” (filed Jun. 7, 2007); U.S. patent application Ser. No. 11/298,339 “RESPIRATORY DEVICES” (filed Dec. 8, 2005); U.S. patent application Ser. No. 11/298,362 “METHODS OF TREATING RESPIRATORY DISORDERS” (filed Dec. 8, 2005); U.S. patent application Ser. No. 11/298,640 “NASAL RESPIRATORY DEVICES” (filed Dec. 8, 2005); U.S. patent application Ser. No. 12/141,875 “ADHESIVE NASAL RESPIRATORY DEVICES” (filed Jun. 18, 2008); U.S. patent application Ser. No. 11/811,401 “NASAL RESPIRATORY DEVICES FOR POSITIVE END-EXPIRATORY PRESSURE” (filed Jun. 7, 2007); U.S. patent application Ser. No. 12/014,060 “METHODS AND DEVICES FOR IMPROVING BREATHING IN PATIENTS WITH PULMONARY DISEASE” (filed Jan. 14, 2008); U.S. patent application Ser. No. 11/941,915 “ADJUSTABLE NASAL DEVICES” (filed Nov. 16, 2007); U.S. patent application Ser. No. 11/941,913 “NASAL DEVICE APPLICATORS” (filed Nov. 16, 2007); U.S. patent application Ser. No. 12/405,837 “NASAL DEVICES WITH NOISE-REDUCTION AND METHODS OF USE” (filed Mar. 17, 2009); U.S. patent application Ser. No. 12/044,868 “RESPIRATORY SENSOR ADAPTERS FOR NASAL DEVICES” (filed Mar. 7, 2008); U.S. patent application Ser. No. 12/369,681 “NASAL DEVICES” (filed Feb. 11, 2009); and U.S. patent application Ser. No. 12/364,264 “CPAP INTERFACE AND BACKUP DEVICES” (filed Feb. 2, 2009). The devices, components, systems, methods and techniques referred to herein may be applied with any of these nasal respiratory devices, alone or in combination.

In general, these devices provide greater resistance to exhalation than to inhalation. These devices are typically “passive” devices, rather than “active” devices. Active devices (such as CPAP machines) apply a positive pressure that may increase resistance to exhalation more than inhalation. Passive devices (as described herein) may be used in conjunction with one or more active devices, but do not themselves actively apply gas or other pressure. Instead, they include or more airflow resistors that inhibit exhalation more than inhalation. These devices may be particularly useful to treat certain breathing disorders such as snoring and sleep apnea, and may be worn by a subject when the subject is either awake or asleep. Indeed, many subjects may apply a nasal device before falling to sleep, so that the device may provide therapeutic benefits during sleep.

Examples of such devices are shown in FIGS. 1A-2B, and briefly described below. Exemplary nasal devices may include an airflow resistor (e.g., a flap valve or multiple flap valves) providing a greater resistance to exhalation than to inhalation, a holdfast to secure the nasal device in communication with the subject's nose, and optionally a rim body forming a passageway in which the airflow resistor is positioned, and an aligner for aligning the device with respect to one or more of the subject's nostrils. These nasal respiratory devices may be configured so that during exhalation through the device (e.g., the opening), the resistance through the device is between about 0.001 and about 0.25 cm H₂O/(ml/sec), and during inhalation through the opening, the resistance through the device is between about 0.0001 and about 0.05 cm H2O/(ml/sec). More particularly, the resistance to exhalation may be between about 0.01 cm H₂O/(ml/sec) and about 0.20 cm H₂O/(ml/sec), and the resistance to inhalation may be between 0.0001 and about 0.02 cm H₂O/(ml/sec). The units (“cm H₂O/(ml/sec)”) may also be written equivalently as cm H₂O*sec/ml or as cm H₂O/ml/sec. Also, these resistances maybe applied to resistance through one or both of a subject's nostrils.

For example, FIGS. 1A and 1B show front and back perspective views (respectively) of one variation of an adhesive nasal device. The nasal device shown in FIGS. 1A and 1B shows two single-nostril devices that have been joined to form a single device. In similar variations the two single-nostril devices are not joined by this bridge region 112, but are kept separate, and may be applied separately. The front view of the nasal device shown in FIG. 1A illustrates the outward-facing side of the nasal device, when it is worn by a subject. FIGS. 1A-2B are examples of nasal devices that may be adapted, modified, or applied as described herein.

Detection and treatment of patients suffering from breathing disorders often requires that the patent's breathing be monitored. Monitoring may be particularly important during treatment, because it allows a physician to estimate the efficacy of treatment, and may permit dynamic modification of the treatment. For example, it may be helpful to monitor respiration in patients suffering from, or at risk for, medical conditions such as snoring, sleep apnea (obstructive, central and mixed), Cheyne Stokes breathing, UARS, COPD, hypertension, asthma, GERD, heart failure, and other respiratory and sleep conditions. Sleep labs may monitor patients to diagnose these and other conditions of sleep disordered breathing. Monitoring typically involves taping a sensor to the subject or applying a mask including a sensor over the subject's nose and/or mouth.

Unfortunately, applying a sensor to a subject in this fashion may be uncomfortable, and may make it even harder for the patient to sleep, confounding the diagnosis and treatment. This may be particularly true when sensors are used in combination with treatments involving a medical device that is worn on the subject's face, nose, and/or mouth. If a separate sensor is used, it may be difficult to match the sensor to the treatment system, which may add to patient discomfort, as the monitoring device and the treatment device must both be worn concurrently. In addition to the loss of comfort, combining sensing and treatment systems may also result in a loss of accuracy, as sensing may interfere with the function of treatment systems. Such problems may persist even with currently available treatment systems that include an integrated monitoring sensor or sensors. In addition, it is often desirable to measure intranasal pressure when a subject is receiving treatment. Measuring pressure intranasally is complicated when the subject is wearing a treatment device, particularly a nasal and/or mouthpiece that is intended to modify respiration.

As mentioned above, positive-pressure (active) devices such as PAP (e.g., CPAP) devices are widely used to treat sleep disordered breathing. PAP devices typically include a mask or nasal pillow which is held against the subject's face, and connected to a device for supplying positive pressure air. PAP devices are active devices, because they actively regulate pressure by providing positive flow. PAP Systems may include sensors to determine respiratory pressure during treatment, which are intended to assess breathing flow rate in the presence of active pressure from the PAP system.

The passive devices (and methods using them for treating breathing disorders) using a passive airflow resistor mentioned above are typically much smaller and lighter and therefore may be more comfortable. These devices are referred to herein as “nasal respiratory devices” or simply “nasal devices”. Examples of these devices may be found in U.S. patent application Ser. No. 11/298,640, titled “NASAL RESPIRATORY DEVICES” (filed Dec. 8, 2005); U.S. patent application Ser. No. 11/298,339, titled “RESPIRATORY DEVICES” (filed Dec. 8, 2005); U.S. patent application Ser. No. 11/298,362, titled “METHODS OF TREATING RESPIRATORY DISORDERS” (filed Dec. 8, 2005); U.S. patent application Ser. No. 11/805,496, titled “NASAL RESPIRATORY DEVICES” (filed May 22, 2007); U.S. patent application Ser. No. 11/811,339, titled “NASAL DEVICES” (filed Jun. 7, 2007); U.S. patent application Ser. No. 11/759,916, titled “LAYERED NASAL DEVICES” (filed Jun. 7, 2007); U.S. patent application Ser. No. 11/811,401, titled “NASAL RESPIRATORY DEVICES FOR POSITIVE END-EXPIRATORY PRESSURE” (filed Jun. 7, 2007); U.S. patent application Ser. No. 11/941,915, titled “ADJUSTABLE NASAL DEVICES” (filed Nov. 16, 2007); and U.S. patent application Ser. No. 11/941,913, titled “NASAL DEVICE APPLICATORS” (filed Nov. 16, 2007). Each of these references is herein incorporated by reference in its entirety.

The pressure sensors appropriate for PAP systems will generally be inappropriate for use with the passive airflow resistor methods and devices mentioned above. For example, FIGS. 1A and 1B illustrate one variation of a nasal respiratory device having a passive airflow resistor. In FIGS. 1C and 1D, the nasal respiratory device includes an airflow resistor 1105 that is positioned in a central opening or passageway through the device. The airflow resistor in this example is a flap valve device. The airflow resistor is configured so that the expiratory airflow through the opening/passageway has a higher resistance than inspiratory airflow. For example, the flap valve 1109 opens virtually completely during inhalation to allow airflow through the device, but remains closed during exhalation (as shown in FIG. 1A). The flap valve is prevented from opening during exhalation by two (or more) flap valve limiters 1111 which at least partially span the opening. The nasal device of FIGS. 1C and 1D also includes two leak pathways 1107, 1107′, which remain open even during exhalation. Careful configuration of the leak pathways and airflow resistors allows the resistance and/or flow rates during inhalation and exhalation to be controlled. For example, a nasal respiratory device may include a resistance to exhalation that is between about 0.01 and about 0.25 cm H2O/ml/sec and a resistance to inhalation that is between about 0.0001 and about 0.05 cm H2O/ml/sec (e.g., when the resistance is measured at 100 ml/sec).

A nasal respiratory device typically also includes a holdfast that secures the device to the nose, so that the airflow resistor is in communication with the nasal passageway. In FIGS. 1C and 1D the holdfast is an adhesive holdfast that extends from the central passageway (opening) and allows the flexible attachment of the device to the nose. Other types of holdfasts, including compressible or compliant holdfasts that at least partially insert into the nose, may also be used.

The opening or passageway of the nasal device shown in FIGS. 1C and 1D is formed within the body of the nasal device; the body of the nasal device is itself formed from an inner body rim 1117 (in FIG. 1D) and an outer body rim 1115 (in FIG. 1C). Other nasal respiratory devices may not include a stiff (or semi-stiff or flexible) rim. The inner body region 1117 may also act as an aligner that helps position the device in the nose.

Nasal respiratory devices such as the nasal device shown in FIGS. 1C and 1D may be used to treat a number of respiratory disorders, including sleep disordered breathing such sleep apnea and/or snoring. However, because these devices are worn over the subject's nose, monitoring breathing while wearing the device may be difficult.

U.S. Ser. No. 12/044,868, titled “RESPIRATORY SENSOR ADAPTERS FOR NASAL DEVICES” (filed Mar. 7, 2008) describes passive nasal respiratory devices such as those shown in FIGS. 1C-1D described above, that are configure to allow a sensor (including a cannula), such as a pressure sensor, so that the device may measure respiration through the device. Thus, these devices may measure flow through the device. Described herein are further modifications for the measurement of intranasal pressure (e.g., pressure within the nose) using nasal respiratory devices, and particularly adhesive nasal respiratory devices that are configured to be secured in communication with the subject's nose and inhibit exhalation more than inhalation. These devices, systems and methods may be used to monitor treatment of a sleep disorder.

Although general descriptions of the passive nasal devices referenced above have been described both functionally and by example in other pending or issued patent applications, some specific variations of nasal respiratory devices have not previously been described. Thus, it would be beneficial to improve upon the devices, kits and methods previously described, and particularly to more fully develop certain embodiments of nasal devices and methods of using, manufacturing, inserting and removing nasal respiratory devices. Described herein are specific variations of nasal devices, accessories for nasal devices, methods of using nasal devices and kits including nasal devices.

In particular, described herein are nasal respiratory devices including a billowing airflow resistor which is one type of airflow resistor which may be used in a nasal respiratory device, including any of those described above. The billowing airflow resistors described herein may be configured to provide a resistance to exhalation that is generally greater than the resistance to inhalation, within the range of resistances to inhalation and exhalation that have been identified as therapeutic by the inventors. Advantages of billowing airflow resistors may include a greater manufacturing and operational tolerances, lower profile, and potentially lower costs of manufacture.

### SUMMARY

The present inventions relate to nasal devices, systems including nasal devices and methods of using, modifying, manufacturing or applying such devices, including method of treatment.

For example, Described herein are devices, methods and systems for sensing and measuring intranasal pressure when a subject is wearing (and may be receiving treatment from) a passive nasal device which is configured to inhibit exhalation more than inhalation. In general, these devices and systems include a sensor on the intranasal side of the device. The sensor may be a cannula or other channel that passes through the nasal device and has an opening on or near the distal end that is placed in communication with the intranasal space and therefore the intranasal pressure. The proximal end of the cannula may extend from the side of the nasal device that faces away from the intranasal space, and may connect (e.g., to connecting tubing or directly) to a pressure transducer for sensing intranasal pressure.

In another example, described herein are nasal respiratory devices adapted for measuring intranasal pressure while secured in communication with a subject's nasal cavity. These nasal devices may include a device body having an opening configured to be placed in communication with the nasal cavity, wherein the device body includes an inner side configured to face the nasal cavity and an outer side configured to face away from the nasal cavity when the device is worn, an airflow resistor in communication with the opening (wherein the airflow resistor is configured to increase the resistance to air exhaled through the nasal device more than the resistance to air inhaled through the nasal device), and a pressure sensor exposed to the inner side of the device and configured to sense intranasal pressure when the device is worn.

As mentioned, the pressure sensor may be a cannula having a distal opening that is configured to communicate with the intranasal region when the device is worn by the subject. The cannula may be connected to a pressure transducer either directly, or via tubing or some other means, so that the pressure may be sensed and/or measured and/or recorded. In some variations, a filter may be included in communication with the lumen of the cannula, e.g., to protect the pressure transducer. In some variations, the pressure sensor may be an electronic pressure sensor, or may include electronic components. The pressure sensor may include the transducer. For example, the pressure sensor including a pressure transducer may be included on the nasal device; a connection (wired or wireless) to a measurement and/or recording device may be included.

The pressure sensor may be configured so that it is not exposed directly to flow through the nasal respiratory device. For example, in variations in which the pressure sensor comprises a cannula, the distal opening of the cannula may be protected from flow through the nasal device by spacing the cannula opening across from a region of the nasal device. In some variations, the cannula opening or sensor region may be a side opening/side sensor that is positioned out of the flow path through the device. In some variations, the cannula opening or other sensor may be protected from the flow path by a filter or buffer.

In general, the nasal device may be a single-nostril nasal device or a whole-nose nasal device. The nasal device may be any of the nasal devices described above in the incorporated patents and patent applications, which is adapted to include the intranasal pressure sensor, as described herein. Thus, any appropriate airflow resistor may be used as part of the nasal device. For example, the airflow resistor may be a flap valve or a plurality of flap valves, a ball valve, an elastic valve, a PEEP or threshold valve, etc. In some variations, the nasal devices include an adhesive holdfast configured to adhesively secure the nasal device at least partly within or at least partly around the subject's nose. In some variations, these nasal device may be part of a CPAP interface device (e.g., U.S. Provisional Patent Application Ser. No. 61/025,582 describing passive nasal device such as those described here which may be used in conjunction with an PAP device). For example, the intranasal pressure sensor element may be positioned so as not to interfere with a PAP device that is used in conjunction with the passive nasal device. In some variations the sensor (e.g., cannula) may extend or be extendable beyond the passive nasal device.

Also described herein are nasal respiratory devices adapted for measuring intranasal pressure while secured in communication with a subject's nasal cavity that include a device body having an opening configured to be placed in communication with the nasal cavity, an airflow resistor in communication with the opening (wherein the airflow resistor is configured to increase the resistance to air exhaled through the opening more than the resistance to air inhaled through the opening), and a cannula passing through the nasal device having a distal opening configured to be exposed to intranasal pressure when the device is worn, wherein the cannula is configured to connect to a pressure transducer for sensing intranasal pressure.

As mentioned, any appropriate nasal device may be used. For example, the nasal device may be an adhesive nasal device including an adhesive holdfast that secures the nasal device to the subject's nose adhesively. The nasal device may include an airflow resistor that is configured as a flap valve or flap valves. In some variations, the cannula passing through the nasal device passes through the airflow resistor portion of the nasal device. For example, the cannula may pass through the flap valve. In some variations the cannula may help secure a portion of the airflow resistor (e.g., the flap of a flap valve in position).

Also described herein are systems for monitoring and/or measuring and/or recording intranasal pressure. For example, described herein are systems for monitoring intranasal pressure that include a passive-resistance nasal respiratory device having an airflow resistor that is configured to inhibit exhalation more than inhalation, an intranasal cannula configured to pass through the nasal respiratory device, and connector tubing configured to connect the intranasal cannula to a pressure sensor. The system may also include a pressure transducer configured to connect to the intranasal cannula via the connector tubing and detect pressure within the lumen of the cannula.

In some variations, the system also includes a filter configured to be placed in communication with the intranasal cannula.

As with any of the device embodiments described herein, the system may include any appropriate nasal device adapted to include the intranasal sensor (e.g., cannula). For example, the passive-resistance nasal respiratory device may include an adhesive holdfast.

In some variations, the devices or systems described herein are included as part of a kit. These kits may include one or more devices, and may include instructions for use. Instructions may be written, visual, or electronic. The instructions may indicate application of the device, connection to one or more sensors (e.g., pressure sensors) and/or recording devices, displays, etc. In some variations, the instructions may include calibration instructions for calibrating the device. The instructions may also indicate any of the method of use steps described herein.

Also described herein are methods of using any of the devices and systems described herein. In general, these methods may include applying the nasal devices described herein to the subject so that the pressure sensor (e.g., cannula or other sensor including a pressure transducer) is exposed to the intranasal space when the device is being worn, and sensing intranasal pressure. The methods may also include steps for connecting the sensor to a transducer and/or recording and/or display device. In some variations, the method includes the steps of calibrating the device to measure intranasal pressure.

For example, described herein are methods of monitoring treatment of a sleep disorder using these devices and/or systems. In some variations a method of monitoring treatment of a sleep disorder includes the steps of securing a nasal respiratory device in communication with the subject's nasal cavity without covering the subject's mouth, wherein the respiratory device includes a passive airflow resistor configured to inhibit exhalation more than inhalation and an intranasal cannula passing through the nasal respiratory device, so that the intranasal cannula communicates with the patient's intranasal space, and monitoring intranasal pressure during respiration using the cannula.

In some variations, the method includes the step of connecting the cannula to a pressure transducer. The cannula may be connected to the transducer via tubing.

Any of the devices, systems and methods described herein may be used in conjunction with other sensors, including sensors adapters, and/or sensors for sensing and measuring flow through the device, or flow out of the device.

Also described herein are are nasal devices and methods of treating a subject using nasal devices. In general, the nasal devices described herein include a billowing airflow resistor that is configured to have a greater resistance to exhalation than to inhalation. The billowing airflow resistor typically includes a first layer that is adjacent to a second layer; the first layer is flexible and billows open during inhalation so that the first layer remains separated from the second layer, but remains substantially parallel to the second layer. During exhalation, the first layer collapses back down against the second layer. Airflow through the device during exhalation may be stopped or may be limited to one or more leak pathways.

For example, described herein are nasal devices configured to inhibit nasal exhalation more than nasal inhalation, and these devices may include: a holdfast configured to secure the device in communication with the subject's nasal cavity; an opening; and a billowing airflow resistor extending across the opening. The billowing airflow resistor may include a first layer, wherein the first layer is flexible, and a second layer adjacent to the first layer, wherein the first layer is configured to lie against the second layer during exhalation through the opening, and further wherein the first layer is configured to separate from the second layer while remaining substantially parallel to the second layer during inhalation through the opening. A first layer that is substantially parallel to a second layer may include edge regions that are not parallel. For example, the edge regions of the first layer may curve upward or away from the second layer, but more central (e.g., non-edge) regions separate from the second layer while remaining substantially parallel to the first layer. The first layer may be constrained (e.g., along two or more sides) to prevent it from opening fully.

Any of these devices may also include one or more leak pathways through the device configured to be open during both inhalation and exhalation. In some variations, no leak pathway is included. Leak pathways may be formed through the airflow resistor (e.g., through both the first and second layers), and/or they may be formed through other regions of the nasal device, including the holdfast.

The billowing airflow resistor may also include a support layer extending across the opening and adjacent to the second layer. For example, the support layer may be one or more struts (e.g., spanning all or a portion of the opening). In some variations the support layer is a mesh layer. In some variations, the second layer is a mesh (and may also be referred to as a support layer, since it supports the first layer and prevents it from opening during exhalation).

In general the first and second layers are oriented so that the second layer prevents the first layer from opening (e.g., separating from the second layer) during exhalation. Thus, the first layer may be configured to be closer to nasal cavity than the second layer.

In some variations, the opening may be at least partially surrounded by the holdfast. For example, the opening may be formed through the holdfast (and may be any appropriate shape, including round, oval, hourglass, etc.). The holdfast may only partially surround the opening. For example, one side may be a leak pathway, between the subject's nose and the airflow resistor.

In general, the holdfast comprises an adhesive layer that is configured to adhesively secure the device to the subject. For example, the holdfast may include a flexible adhesive holdfast layer that extends in substantially the same plane as the billowing airflow resistor before application to a subject's face. The holdfast may include a biocompatible adhesive. The holdfast may also be configured to secure the device in the subject's nose, partially in the nose, or over the subject's nose, or any combination thereof. For example, the holdfast may be a compressible holdfast that expands to fit the subject's nose, or is otherwise compliant with the subject's nose.

The first layer is typically flexible, and may be made of a flexible and/or elastic material. The thickness of this first layer may also be controlled to help determine the flexibility, and thus the resistance or operation of the device. In some variations, the first layer is made of a 1 mil (0.001 inch) thick polyurethane. In addition, the first layer of the billowing airflow resistor may have any appropriate shape that allows it to billow (separate from the second, adjacent layer while remaining substantially parallel to the second layer). For example, the first layer may comprise a plurality of strips extending across the opening. In some variations, the first layer is secured at or near the edge of the opening. For example, the first layer may be secured at two areas across the opening, leaving two edges that are allowed to “open” when the layer billows. In some variations the first layer is loose, so that the material may be less stretchable (or relatively non-stretchable), but the first layer may separate from the second layer to allow passage of air. For example, the first layer may be folded or creased so that it can expand during inhalation, and separate from the second layer.

In some variations flow between the first and second layers during inhalation occurs past one or more edges of the first layer. In some variations, the first layer includes one or more (e.g., a plurality) of openings there through.

The second layer may be a relatively stiff material. For example, the second layer may be formed of a polycarbonate material (e.g., 0.009 inch thick sheet polycarbonate) that includes one or more openings. In some variations, the second layer is a mesh.

In general, the first and the second layers include offset openings, through which air can flow during inhalation, when the first and second layers are separated slightly, but which are closed when the two layers are adjacent to each other during exhalation. For example, the first layer may include one or more openings that are offset from openings in the second layer when the first and second layers lie against each other during exhalation.

The billowing airflow resistors are generally configured to have an appropriate range of resistance to exhalation and inhalation during operation. For example, leak pathway may be adjusted to adjust the resistance to exhalation. A billowing airflow resistor may be configured so that during exhalation through the device, the resistance through the device is between about 0.001 and about 0.25 cm H₂O/(ml/sec), and during inhalation through the opening, the resistance through the device is between about 0.0001 and about 0.05 cm H2O/(ml/sec). More particularly, the resistance to exhalation may be between about 0.01 cm H₂O/(ml/sec) and about 0.20 cm H₂O/(ml/sec), and the resistance to inhalation may be between 0.0001 and about 0.02 cm H₂O/(ml/sec). The units (“cm H₂O/(ml/sec)”) may also be written equivalently as cm H₂O*sec/ml or as cm H₂O/ml/sec. The resistances described above were estimated at a flow rate of 100 ml/sec (resistance may be dependent on flow rate). Also, these resistances may be applied to resistance through one or both of a subject's nostrils.

Also described herein are nasal respiratory devices configured to inhibit nasal exhalation more than nasal inhalation, that include a holdfast configured to secure the device in communication with the subject's nasal cavity, an opening, and a billowing airflow resistor extending across the opening, where the billowing airflow resistor comprises: a flexible layer secured across the opening, and a sealing face layer adjacent to the flexible layer, and wherein the billowing airflow resistor is configured so that during exhalation through the opening, the flexible layer presses against the sealing face layer; and during inhalation through the opening the flexible layer separates slightly from the sealing face layer while remaining substantially parallel to the sealing face layer.

Any of the features previously described for nasal devices including billowing airflow resistors may be included. For example, the devices may include a leak pathway through the device configured to be open during both inhalation and exhalation.

The holdfast may include an adhesive layer configured to adhesively secure the device to the subject. In some variations, the opening is at least partially surrounded by the holdfast. The holdfast may comprise a flexible adhesive holdfast layer that extends in substantially the same plane as the billowing airflow resistor.

The flexible layer of the billowing airflow resistor may include a plurality of strips extending across the opening. The flexible layer may be secured across the opening in at least two regions. For example, the flexible layer may include a plurality of openings there through. The sealing face layer may be formed of a relatively stiff material. The sealing face layer may be a second layer of the billowing airflow resistor, as described above. The flexible layer may include one or more openings that are offset from openings in the sealing face layer when the flexible layer and the sealing face layer press against each other during exhalation.

As mentioned above, the billowing airflow resistor may be configured so that during exhalation, the resistance through the device is between about 0.001 and about 0.25 cm H₂O/(ml/sec), and during inhalation through the opening, the resistance through the device is between about 0.0001 and about 0.02 cm H2O/(ml/sec).

Also described herein are nasal respiratory devices configured to inhibit nasal exhalation more than nasal inhalation, the device comprising: a holdfast configured to secure the device in communication with the subject's nasal cavity; an opening at least partially surrounded by the holdfast; and a billowing airflow resistor extending across the opening. The billowing airflow resistor may include: a first layer, and a second layer adjacent to the first layer, wherein the first layer is configured to lie against the second layer during exhalation through the opening to provide a resistance of between about 0.001 and about 0.25 cm H₂O/(ml/sec) through the device, and further wherein the first layer is configured to separate from the second layer during inhalation through the opening, while remaining substantially parallel to the second layer to provide a resistance of between about 0.0001 and about 0.02 cm H2O/(ml/sec) through the device.

Methods of using a nasal device including a billowing airflow resistor are also described. For example, described herein are methods of treating a respiratory disorder including the steps of: securing a nasal device in communication with the subject's nasal cavity, wherein the nasal device comprises a billowing airflow resistor having a flexible layer and a sealing face layer; driving the flexible layer against the sealing face layer during exhalation to create a resistance to exhalation through the nasal device; and separating the flexible layer from the sealing face layer during inhalation while maintaining the layers substantially in parallel to create a resistance to inhalation through the nasal device that is less than the resistance to exhalation.

The resistance to exhalation may be between about 0.001 and about 0.25 cm H₂O/(ml/sec). The resistance to inhalation may be between about 0.0001 and about 0.02 cm H2O/(ml/sec). The step of securing the nasal device may include adhesively securing the device over both of a subject's nostrils. The step of securing the nasal device may also include securing the nasal device at least partially within a subject's nostril.

In general, the devices described herein may be used to treat respiratory disorders such as sleeping disorders, including those selected from the group consisting of: snoring and sleep apnea.

Also described herein are adapters that may be used with the passive nasal devices mentioned or described herein. An adapter may be used to act as an intermediate interface between a passive nasal device and a subject's nose or nostrils. For example, an adapter may be a nose mask to which one or more passive nasal devices can be attached. Thus, if the passive nasal devices are otherwise single-use or disposable devices, the adapter may allow them to be re-used. In some variations an adapter is a replacement holdfast portion that may be applied to replace or supplement a holdfast of a passive nasal device. For example, a ring of adhesive material (forming a replacement holdfast) may be applied to the holdfast of a passive nasal device, e.g., after the passive nasal device has been worn at least once and removed.

For example, described herein are nasal adapter devices for use with a disposable nasal device, the adapter device comprising: a housing configured to be secured to a subject's nose without covering the subject's mouth; an opening through the housing, the opening having a perimeter region that is configured to secure a passive nasal device thereto so that the passive nasal device may inhibit exhalation more than inhalation through the subject's nose when the passive nasal device is secured to the perimeter region; and a holdfast configured to seal the housing to the subject's nose so that substantially all of the airflow to and from the subject's nose passes through the disposable passive nasal device secured to the perimeter region.

The housing may be a nose mask. For example, the nose mask may be configured to fit over the subject's nose and be sealed against the nose. The opening may be positioned across from one or both of the subject's nostrils. The nasal mask may seal against the subject's face, so that air passing into/out of the nose must pass through the opening (or a passive nasal device attached over or in the opening. Thus, in some variations, the housing may include a sealing edge region configured to secure the edge of the housing to a subject's face. As mentioned, the opening through the housing is located opposite to a subject's nostril or nostrils when the adapter device is worn by the subject.

Any appropriate holdfast may be used as part of the adapter. For example, the nose mask adapter holdfast may include a strap. In some variations, holdfast comprises an adhesive. The holdfast may also include a compressible sealing ring around the perimeter of the housing, which may help form the seal to the subject's face.

The adapter device may also include a passive nasal device configured to inhibit exhalation more than inhalation.

Also described herein are methods of applying a disposable passive nasal device to an adapter nose mask, wherein the passive nasal device is configured to inhibit exhalation more than inhalation, the method comprising: exposing a perimeter region around an opening through an adapter nose mask, wherein the adapter nose mask further comprises a holdfast for sealing the adapter nose mask over or in the subject's nose without substantially covering the subject's mouth; and securing the holdfast of the passive nasal device over or across the opening through the adapter nose mask so that when the adapter nose mask is worn by a subject, substantially all of the air flowing into and out of the subject's nose passes through the passive nasal device, to inhibit exhalation more than inhalation.

The method may also include the steps of securing the adapter nose mask over the subject's mouth. The step of securing the holdfast of the passive nasal device over or across the opening through the adapter nose mask may include adhesively securing the passive nasal device to the adapter nose mask. In some variations, the step of securing the holdfast of the passive nasal device over or across the opening through the adapter nose mask comprises expanding a compressible holdfast of the passive nasal device at least partially within the opening through the adapter nose mask.

Also described herein are methods of applying an adapter to a passive nasal device so that the passive nasal device may be re-used, wherein the passive nasal device comprises an airflow resistor configured to inhibit exhalation more than inhalation and an adhesive holdfast, the method comprising applying a replacement adhesive holdfast over the adhesive holdfast of the passive nasal device.

In addition to the inventions summarized above, various other embodiments are described in greater detail below.

### BRIEF DESCRIPTION OF THE DRAWINGS

FIGS. 1A and 1B are bottom and top perspective views, respectively, of one variation of a nasal device. FIGS. 1C and 1D show bottom (external) and top (internal) perspective views of another variation of a nasal respiratory device.

FIGS. 2A and 2B show one variation of a layered nasal device in a top view and an exploded perspective view, respectively.

FIG. 3 shows one variation of a nasal device having a non-moving airflow resistor.

FIGS. 4A-4C illustrate one variation of a nasal device configured to change the size of the nasal passages and/or openings.

FIGS. 5A-5C illustrates a nasal device having a rotating airflow resistor.

FIG. 6 shows a nasal device including a sensor.

FIGS. 7A and 7B show side and top views, respectively, of an airflow resistor for a nasal device including a valve activating/disabling element.

FIG. 8 schematically illustrates one variation of a method for applying and/or operating an adhesive nasal device.

FIG. 9 illustrates an airflow resistor including a valve stiffening element.

FIGS. 10A-10C illustrate another variation of an airflow resistor as described herein.

FIG. 11 illustrates a face/nose mask configured for use with a nasal device as described herein.

FIG. 12 illustrates the application of a nasal device configured as a deep-nostril implant.

FIGS. 13A and 13B illustrate a nasal device having an adhesive holdfast that attaches to the bridge of the nose.

FIG. 14 describes a nasal device configured as a septal clip.

FIG. 15 shows and illustrates operation of a nasal device adapter.

FIGS. 16A-16C illustrates another variation of a nasal device adapter.

FIGS. 17A and 17B show bottom (external) and top (internal) perspective views of one variation of a nasal respiratory device including a cannula for sensing intranasal pressure.

FIG. 17C shows a side perspective view of the device shown in FIGS. 17A and 17B.

FIG. 18 shows a partial perspective view of the device shown in FIGS. 17A-17C.

FIGS. 19A and 19B show partial perspective views of a portion of a nasal device including a cannula for sensing intranasal pressure.

FIGS. 20A and 20B show rotated views (rotated by 90°) of the partial perspective views shown in FIGS. 19A and 19B, respectively.

FIG. 21 shows a top perspective view of the top of a nasal respiratory device including cannula for sensing intranasal pressure.

FIG. 22 illustrates a cross-section through a nasal respiratory device including cannula for sensing intranasal pressure.

FIG. 23 shows an exploded view of the pair of nasal devices shown in FIGS. 24A-24B.

FIGS. 24A and 24B illustrate a pair of nasal devices including sensors for sensing intranasal pressure.

FIG. 25 shows a system for sensing intranasal pressure including a nasal device configured to inhibit exhalation more than inhalation.

FIG. 26 shows a perspective view of another system for sensing intranasal pressure, as described herein.

FIG. 27 illustrates a method of monitoring intranasal pressure.

FIG. 28A is a cross-section through one variation of a nasal device having a billowing airflow resistor.

FIG. 28B is a perspective view of the device shown in FIG. 28A.

FIG. 28C is an exploded view of a nasal device including a billowing airflow resistor.

FIG. 28D is a perspective view of the device of FIG. 28C.

FIG. 29A is a top view of a first layer of one variation of a billowing airflow resistor and FIG. 29B is a top view of a second layer of one variation of a billowing airflow resistor.

FIG. 29C shows a partial view of a billowing airflow resistor including the first and second layers of FIGS. 29A-29B.

FIGS. 29D and 29E are a cross-sectional views through the billowing airflow resistor shown in FIG. 29C during exhalation and inhalation, respectively.

FIG. 30A is a top view and FIG. 30B is a perspective view of another variation of a billowing airflow resistor.

FIG. 31 is a partial exploded view of another variation of a billowing airflow resistor.

FIGS. 32A and 32B illustrate a first and second layer of a billowing airflow resistor, respectively, and FIG. 32C shows an exploded view of a billowing airflow resistor which may be included as part of a nasal device.

FIGS. 33A and 33B illustrate a first and second layer of another variation of a billowing airflow resistor, respectively, and FIG. 33C shows an exploded view of a billowing airflow resistor which may be included as part of a nasal device.

FIGS. 34A and 34B illustrate a first and second layer of another variation of a billowing airflow resistor, respectively, and FIG. 34C shows an exploded view of a billowing airflow resistor which may be included as part of a nasal device.

FIGS. 35A and 35B illustrate a first and second layer of another variation of a billowing airflow resistor, respectively, and FIG. 35C shows an exploded view of a billowing airflow resistor which may be included as part of a nasal device.

FIG. 36 illustrates airflow pathways through the exploded view of the airflow resistor shown in FIG. 35C during inhalation.

FIG. 37A is an exploded view of a nasal device including a billowing airflow resistor.

FIG. 37B is a top view of the nasal device shown in FIG. 37A.

FIG. 38A shows one variation of a flexible layer of a billowing nasal device; FIG. 38B shows one variation of a second (e.g., sealing face) layer configured for use with the flexible layer shown in FIG. 38A.

FIG. 38C shows the flexible (first) layer adjacent to the sealing face (second) layer, the first layer has been made transparent.

FIG. 39 is an exploded view of another variation of a nasal device including a billowing airflow resistor having the first layer shown in FIG. 38A and the second layer shown in FIG. 38B.

FIGS. 40A and 40B illustrate operation of a nasal device with a billowing airflow resistor as shown in FIGS. 38A-38C and 39.

FIG. 41A is a side view of a billowing airflow resistor for a nasal device during exhalation; FIG. 41B is a side view of the same airflow resistor during inhalation. FIG. 41C is a top view of the airflow resistor shown in FIGS. 41A and 41B.

FIG. 42 is an exploded view of another variation of nasal device having both billowing and flap-type airflow resistor valves.

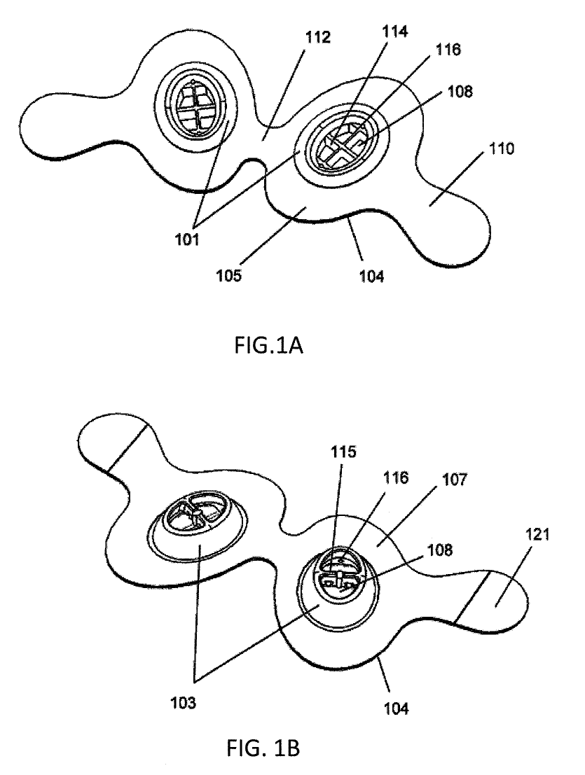

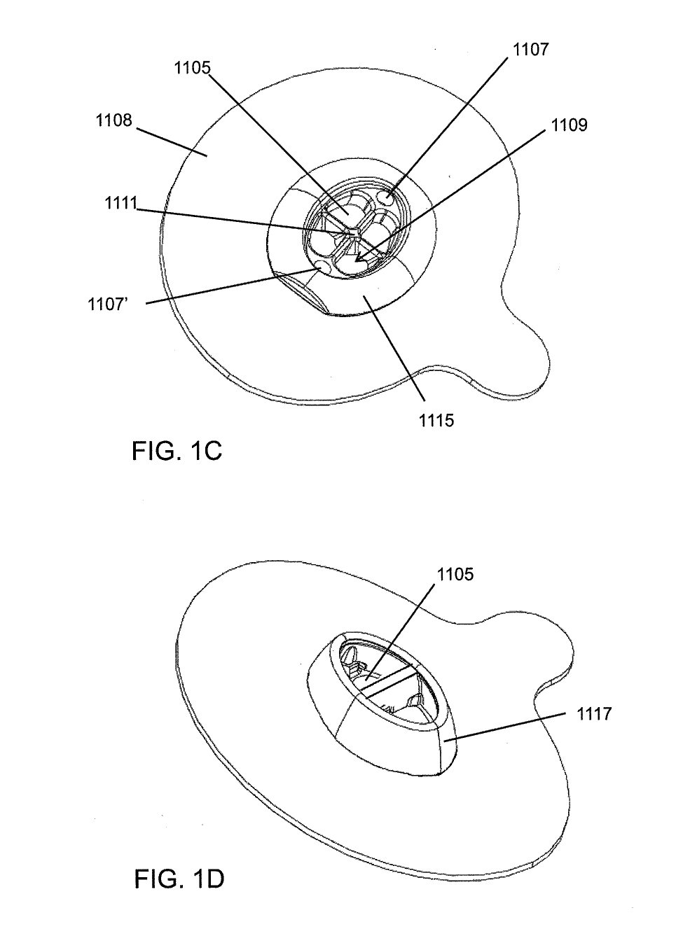

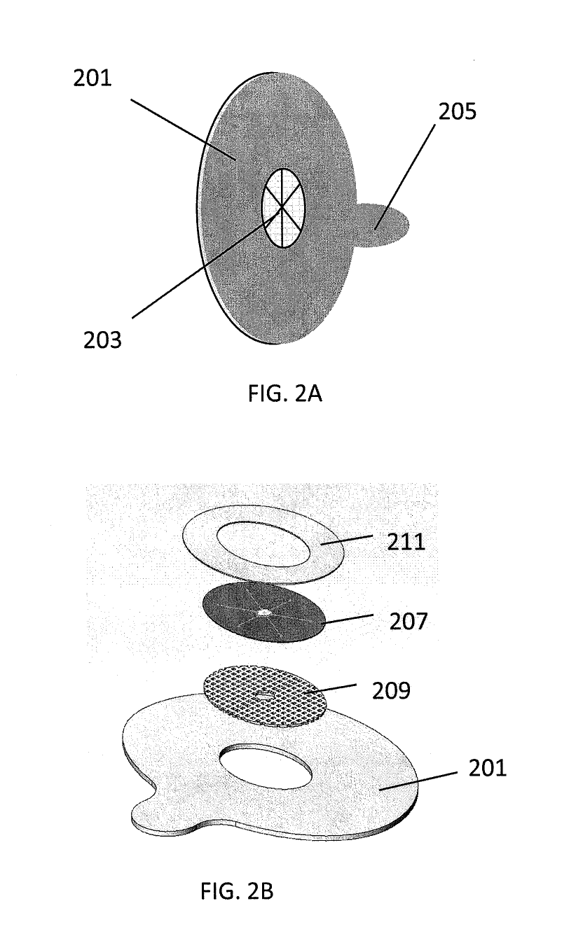

### DETAILED DESCRIPTION

As used herein, a nasal device inhibits exhalation more than inhalation. For example, a nasal device may provide an increased resistance to exhalation compared to inhalation. Thus, a nasal device may be configured so that during exhalation, the resistance to exhalation is between about 0.001 and about 0.25 cm H₂O/(ml/sec), and during inhalation the resistance is between about 0.0001 and about 0.05 cm H₂O/(ml/sec). More particularly, the resistance to exhalation may be between about 0.01 cm H₂O/(ml/sec) and about 0.20 cm H₂O/(ml/sec), and the resistance to inhalation may be between 0.0001 and about 0.02 cm H₂O/(ml/sec).

In some variations, the nasal device includes an airflow resistor, as previously described and characterized in detail in the reference incorporated above. An airflow resistor may include a movable member such as a flap, or non-movable members. For example, an airflow resistor configured to increase resistance to exhalation more than inhalation may include a non-valved or non-movable member.

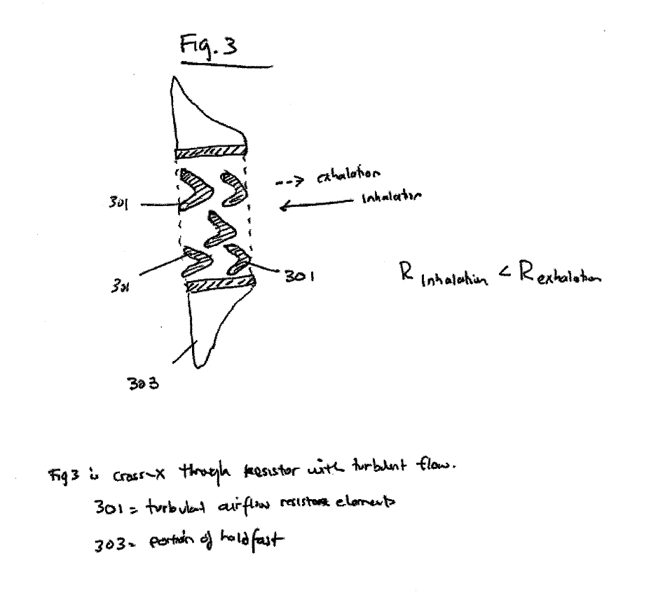

In some variations, the airflow resistor increases the turbulence preferably during exhalation and thereby increases the resistance to exhalation. For example, increase turbulent flow during exhalation compared to inhalation (e.g., turbulent in exhalation, laminar in inhalation) will result in greater resistance to exhalation (Rₑ) compared to the resistance to inhalation (Rᵢ). FIG. 3 illustrates one variation of a nasal device including an airflow resistor that is configured to selectively increase turbulence during exhalation, and thereby increase the resistance to exhalation. In this example the airflow resistor region is positioned within a central passageway or opening through the nasal device, and includes a plurality of turbulence airflow elements 301. In this example, the elements are shown in cross-section as having a concave face exposed to the inside of the nasal passage and a smooth, more aerodynamic face in the opposite direction. As indicated by the arrows, during exhalation, airflow will push against the concave surfaces, and will be turbulent. During inhalation, the airflow will stream past the smooth surfaces on the opposite side, and will be substantially less turbulent or even laminar. Other variations of airflow resistors may be shaped similar to an airplane wing, which has also induces turbulent flow in one direction and laminar flow in the opposite direction.

An airflow resistor configured to increase turbulence in this manner may be non-moving (e.g., fixed), as described above. Airflow resistors which increase turbulence may also include movable elements for selectively increasing turbulence, or a combination of fixed and movable elements.

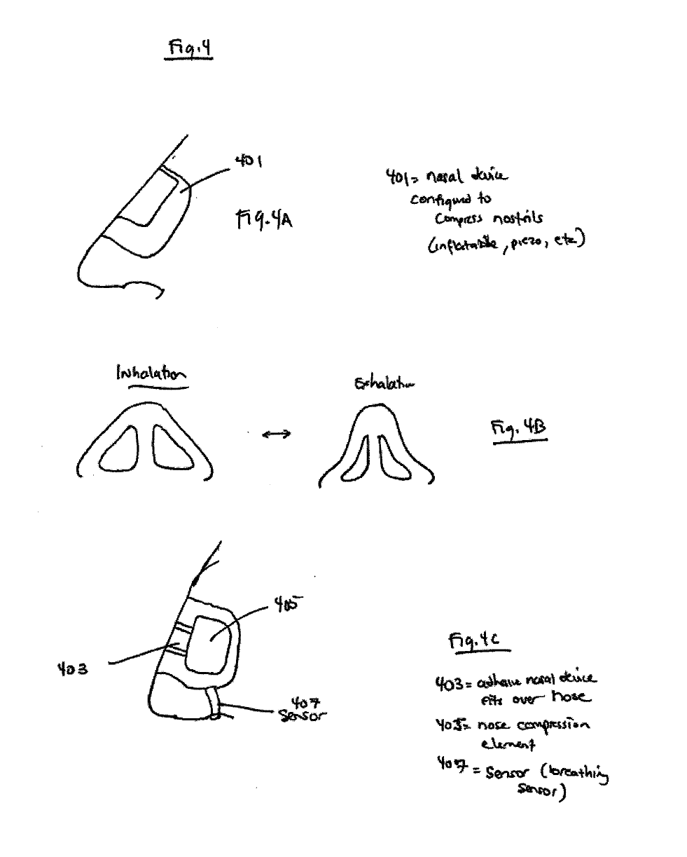

Some nasal devices may not include an occlusive or partially occlusive airflow resistor, but may incorporate a portion of the subject's nose, nostrils, or nasal cavity to increase the resistance to exhalation compared to inhalation. For example, in some variations the nasal device constricts the nose or nostril openings during all or a portion of the exhalation phase or respiration. In some variations the nasal device mechanically constricts the nose or nostril openings, as illustrated in FIGS. 4A-4C. FIG. 4A shows a nasal device that mechanically constricts the nostril opening from on top or in front of the nose. The nasal device may be placed over all or a portion of the nose (e.g., spanning the bridge of the nose, as shown in FIG. 4A), and the device squeezes the nose to constrict the nostril openings, as indicated in FIG. 4B. The constriction may be achieved by pinching or compressing a portion of the nose. In some variations the device includes a mechanical member that can be actuated to compress the nose. For example, the device may include a piezoelectric member that can be actuated to compresses the nose. In some variations, the device includes one or more sensors for detecting respiratory cycle. Alternatively a separate member may be used to determine the respiratory cycle. The device may then be controlled (e.g., via a controller running control logic) to actuate during exhalation. Sensors for determining all or a portion of the respiratory cycle are described in greater detail below, but may include gas sensors (e.g., CO2 sensors, oxygenation sensors, etc.) airflow sensors, lung position sensors, movement sensors, etc. The sensor may be attached to the device, as shown in FIG. 4C, or it may be linked by telemetry, and may be located remote to the nasal device.

Other device may constrict the nasal opening by residing within (or at least partially within) the nose. For example, the nasal device may include one or more inflatable elements that inflate during exhalation to partially (or fully) occlude the nostril or nostril opening.

Other non-moving airflow resistors may be used as well. For example, the nasal device may include a membrane or barrier that is more permeable in one direction than another. In some variations the device may include swelling/swellable materials that swell during a portion of the expiratory cycle to increase resistance during exhalation. For example, airflow resistor may include a bi-stable material that transitions from a less occluding position to a more occluding position based on the moisture or temperature which is different during exhalation than inhalation.

A nasal device may be configured to affect expiratory pause, which is the pause between inhalation and exhalation. This may be effected by changing the resistance to exhalation and/or inhalation, as mentioned, or it may be accomplished by changing the mechanical resistance to breathing. The mechanical resistance may be altered at the chest/diaphragm level. For example, a device may include a chest strap that can selectively constrict the chest to increase the mechanical resistance to breathing. This may be done even in the absence of a device attached to the subject's nose.

In some variations, movable airflow resistors are used as part of a nasal device which rotates. FIGS. 5A-5C illustrate a nasal device including a rotating airflow resistor configured to include a propeller. A propeller-type airflow resistor may be rotated freely, or relatively freely, during inhalation, but may have a resistance to rotation during exhalation. For example, the propeller-type device may rotate slightly during inhalation in a first direction until it can freely rotate; during exhalation the propeller may be rotated back down at greater resistance.

FIG. 5A shows a propeller-type region of an airflow resistor. In this device the propeller is a spinner valve that is configured to rotate in a first direction during inhalation and the opposite direction during exhalation.

In FIG. 5B, the propeller region is attached to the rest of the airflow resistor, and is shown in cross-section. The spinner/propeller rotates around a pivot center 503, and the resistance to rotation is greater in the direction of airflow during exhalation than during inhalation. For example, the pivot center may include resistive elements that mechanically interfere with rotation in only one direction. Alternatively, the walls of the passageway in which the spinner sits may increase the resistance during rotation in one direction more than the other. A cross-section through a nasal device including such an airflow resistor is shown in FIG. 5C.

As mentioned above, any appropriate sensor or sensors may be used. Nasal devices including one or more sensors may be configured to sense and/or determine the respiratory cycle point, the onset of sleep, or the onset of snoring. This information may be used to control the operation of a nasal device. In some variations the resistance provided by the nasal device may be altered based on the respiratory cycle, e.g., to increase the resistance to exhalation. A device for sensing respiratory cycle may include a CO₂ sensor, since the level of CO₂ is typically higher during exhalation. One or more airflow sensors may also be used. An airflow sensor may include a temperature sensor (e.g., thermister) or the like, or a pressure sensor, or a moisture sensor. The sensor may be attached or connected directly to the nasal device, as illustrated in FIG. 6, or it may be located elsewhere on the subject, such as the subject's most, throat, etc. A sensor may communicate wirelessly or via a wire to the nasal device. The nasal device may include a controller, which may run control logic, to interpret the data from the sensor(s) and to regulate the airflow resistor. In some variations the device analyzes and responds to this sensor information. In some variations the device may record and/or transmit the sensor information. Other sensors may include lung position sensors, which may also help determine the respiratory cycle. In some variations, a motion or vibration sensor may be used. For example, a vibration sensor may be configured to detect sound and/or movement associated with snoring. In this manner the sensor may be used to activate (e.g., “turn on”) the resistance to exhalation. This may be accomplished by engaging or disengaging the airflow resistor. For example, a device may include a disengaging element that may inactivate the airflow resistor so that the resistance during exhalation remains low. FIGS. 7A and 7B illustrate one variation of such a device, including a controller for controlling the device.

In FIG. 7A, the device includes a valve disabling element 709 that disengages the flap valve. A controller 713 may control this disabling element, and may receive input from a sensor, such a snore-detecting (vibration) sensor. In one variation, the device inactivates the flap valve (e.g., by holding it open) until a vibration or sound indicative of a snore is heard, then the controller instructs the valve disabling element to release the flap valve, and allow it to increase the resistance to exhalation. Once activated the device may remain active (inhibiting exhalation more than inhalation), or it may remain on for some time period (“on demand” activation). In some variations the controller includes a timer, and may activate, disengaging the valve disabling element, after some predetermined time period (e.g., perhaps allowing the subject to begin sleeping).

In some variations, the nasal device may be configured to activate (inhibiting exhalation more than inhalation) during a particular sleep state (e.g., REM, deep sleep states, etc.).

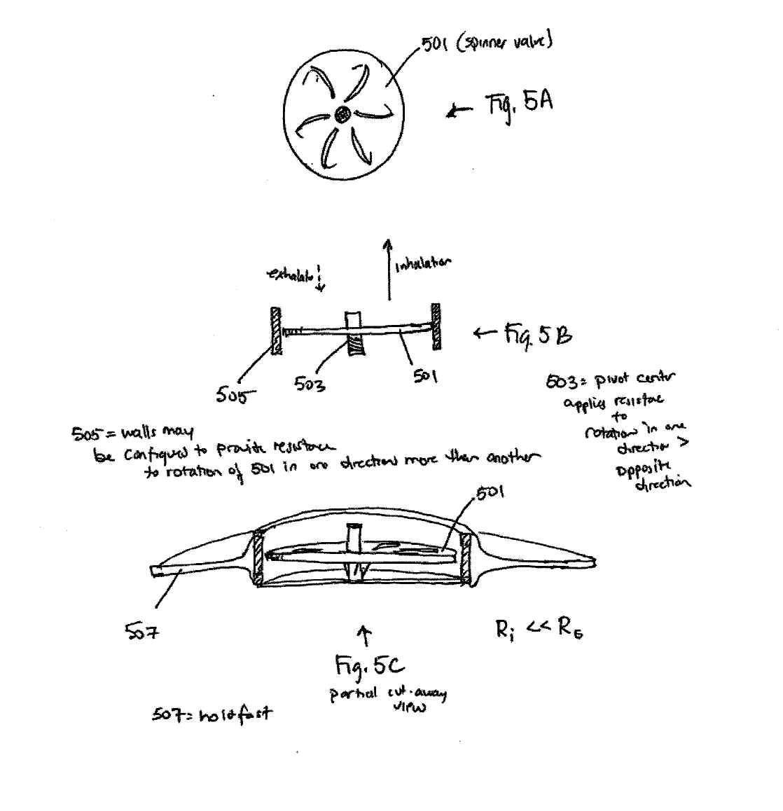

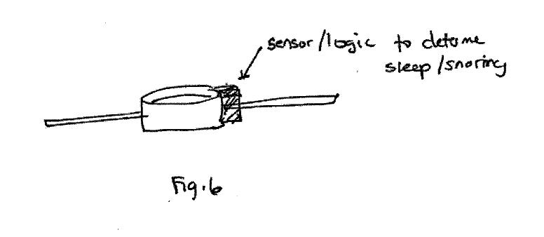

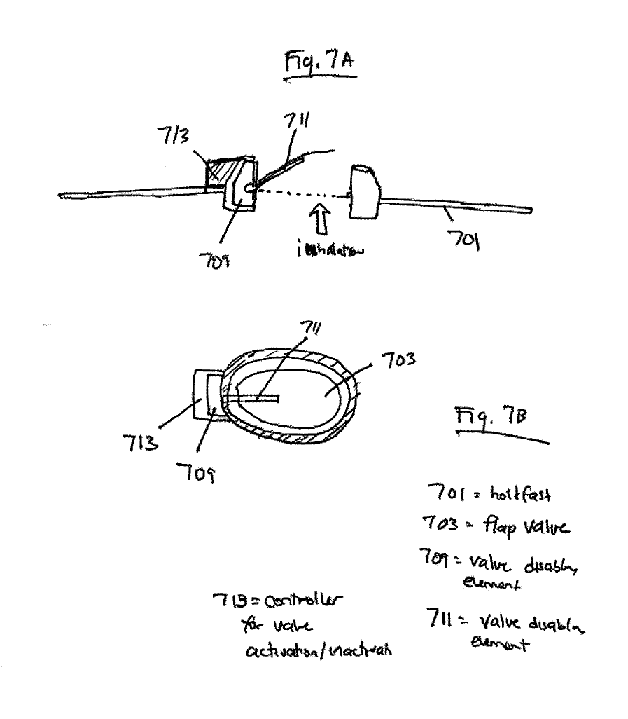

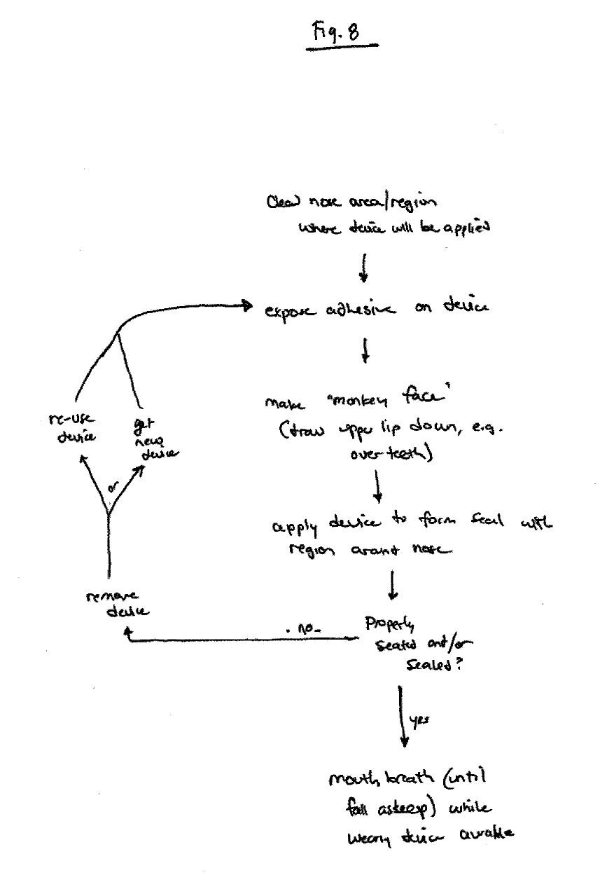

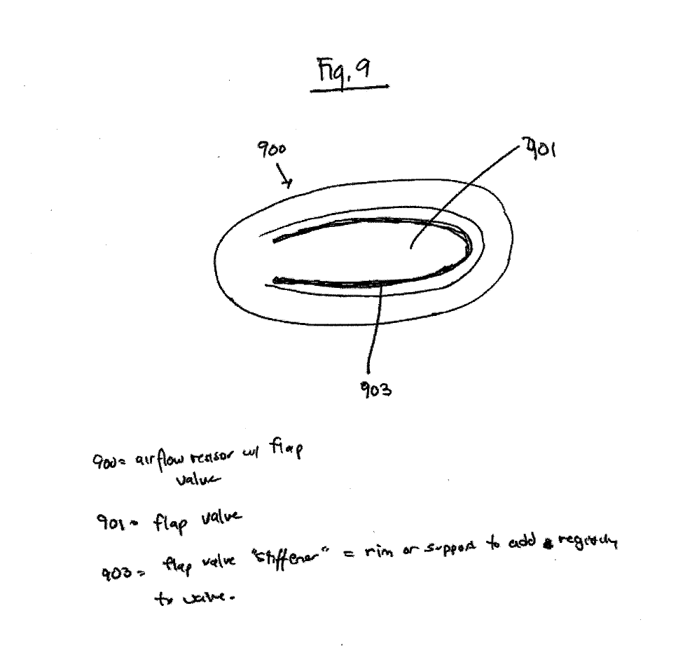

In operation, any of the devices described herein may be used to treat a disorder such as sleep apnea (e.g., obstructive sleep apnea) and/or snoring. The method of use may include the step of instructing the subject how to apply, wear and use the device. For example, a method of using an adhesive-type nasal device is outlined in FIG. 8. Any of the steps in this example may be option, and additional steps may be added. For example, the subject may be told to clean the area that the adhesive nasal device is to be applied to, such as the skin around the nostrils. Cleaning may be performed by wiping, or by using a cleanser such as soap and/or alcohol. Cleaning may help remove oils or other debris that might inhibit forming a seal by the adhesive holdfast.

After cleaning, the protective cover may be removed from the nasal device(s) in variations including such a protective cover, to expose the adhesive holdfast. In some variations the device may be wetted to activate the adhesive holdfast. The subject may then be instructed to make an expression (e.g., “monkey face”) that will help expose the skin region to the adhesive holdfast. For example, a “monkey face” expression is made by extending the upper lip downward, e.g., over the teeth (upper teeth); the lower jaw may also be dropped, elongating the face. The lips may be closed together. Thereafter, the adhesive may be secured to the nose, and sealed around the nostril opening(s). The adhesive may then be tested to determine if a seal has been made. Testing may be performed by touch (e.g., feeling for creases, etc.) or by attempting a strong exhale through the nose. If the device is not properly seated or sealed, it may be removed, and reapplied, or a new device may be applied, as before.

A subject wearing the device may also be instructed to mouth breathe. This instruction is counterintuitive, but may be especially helpful to enhance the comfort and usefulness of the device. The range of resistance indicated above (e.g., a resistance to exhalation is between about 0.001 and about 0.25 cm H₂O/(ml/sec), and during inhalation the resistance is between about 0.0001 and about 0.02 cm H2O/(ml/sec)) for many of the nasal devices described may be difficult for a subject to quickly acclimate to, particularly if the subject is trying to sleep. However, once the subject is asleep, most people will desist mouth breathing and switch to nose-breathing through the device, even at these resistance ranges. Thus, instructing a subject to mouth breath may allow the subject to more comfortably fall asleep while still benefiting from the increased resistance to exhalation during sleep.

As a result of the instructions to mouth-breath immediately after applying the device, the subject may also be instructed to take one or more steps to prevent or treat dry mouth, which may result. In some variations, as system including a nasal device may including instructions and/or therapeutics for treating dry mouth. For example, a subject may be instructed to keep their mouth moist by sipping water or other sugar-free juices frequently, prior to sleeping (e.g., including keeping a water bottle, or glass of water may be kept at the bedside). Also, a humidifier may be used to increase the moisture content of the air when sleeping. In some variations, particularly in instances in which a particularly subject is especially susceptible to dry mouth, the nasal devices may be taken in conjunction with a pharmaceutical or neutriceutical agent to increase the flow of saliva. For example, drugs such as Pilocarpine (Salagen™) and Cevimeline (Evoxac™) may be used to with the devices to reduce the sensation of oral dryness. Other salivary stimulants, oral moisturizers and salivary substitutes may also be used in conjuction with the devices described herein. For example, gum (chewing gums) may be recommended or included. Chemical stimulation (e.g., Mucopolysaccaharide Sol., citric acid, etc.), and/or electrical stimulation (e.g., “Salitron,” an intra-oral electronic stimulator of saliva, Biosonics, Inc.), and/or pharmacologic stimulants such as cholinergic agonists may also be used in conjunction with these nasal devices. Oral moisturizers and/or salivary substitutes (e.g., Salivart, Oralube, Xero-Lube, Plax, Oral Balance, etc.) may be used as well.

In addition, the subject may be instructed to lay in one or more preferred body positions. For example, the subject may be instructed to initially sit up or lie prone.

A method of removing the nasal device may also be provided. For example, a method may include providing instructions on how to remove the device. Removal may include applying humidity or moisture. For example, the instructions may tell a subject to remove the device (particularly an adhesive device) in a humid environment such as the shower.

In some variations, the device may be configured to prevent wrinkling or disruption of the device, including the holdfast and/or the airflow resistor. For example, wrinkling of the devices may occur as the device is worn or operated. In particular, wrinkling of the flap valve region in variations including flap valves may otherwise interfere with the resistances and operation of the device. FIG. 9 illustrates one variation in which the flap valve includes a stiffener to help prevent wrinkling of the flap valve as it is operated and worn. In this variation the stiffener is a wire, bead, or thickening, preferably peripherally, on the flap valve. Other variations include regions of different material properties or materials that help stiffen the area around the valve.

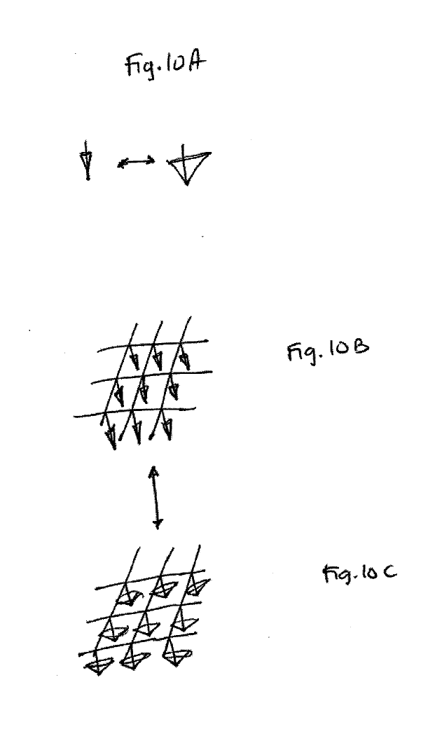

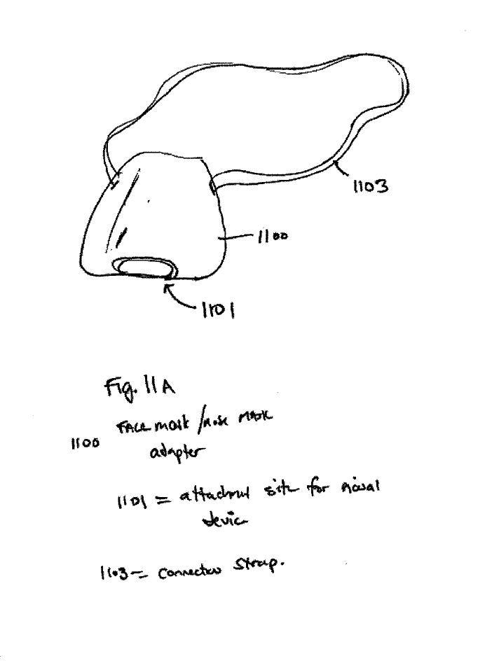

In addition to the airflow resistors previously described, in some variations, the airflow resistor includes one or more expandable member that expands during or before exhalation to increase the resistance. For example, the airflow resistor may include an “umbrella”-type expandable member, as illustrated in FIGS. 10A-10C. In this variation, the umbrella element may be formed of a polymeric material, and may include a membrane that expands from a closed configuration to an open one, as shown in FIG. 7A. FIG. 7B shows an portion of an airflow resistor including a plurality of small expandable umbrella-like members in the close state (e.g., during inhalation). During exhalation, as shown in FIG. 7C, the umbrella-like structures may expand, at least partially blocking airflow.

In some variations, the airflow resistor includes a metering valve that has a continuously variable resistance through the device during exhalation (and/or inhalation).

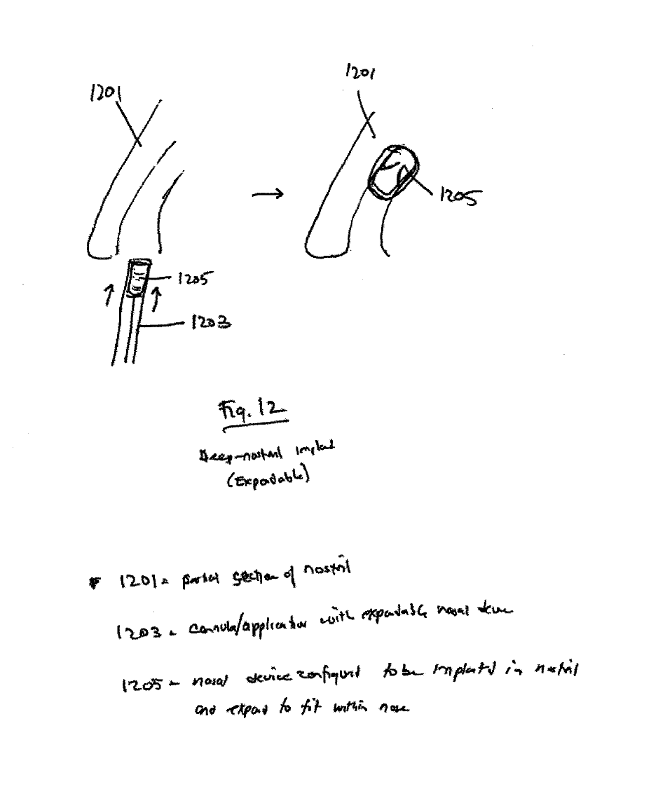

A nasal device may generally be attached to a subject in any appropriate manner. Adhesive nasal devices and expandable nasal device have been previously described. In addition to these, in some variations the nasal device is not secured directly to the subject, but is attached to another mask or adapter that is secured to the subject. For example, FIG. 11 illustrates a nasal mask that is configured to be used with a nasal device. The nasal mask includes an opening near the nostril openings where an adhesive nasal device may be secured. The opening may be adapted for use with one particular configuration of nasal device (e.g., a layered whole-nose nasal device). The nasal mask may be secured over the subject nose. For example, the nasal mask may be secured over the subject's nose by the band or strap 1103.

Mouth masks, face masks, or mouth/nose masks may also be used. One advantage to such adapters for use with these nasal devices is that they may be reused, or may allow re-using of the nasal device more readily, since the nasal device does not have to be removed from the face.

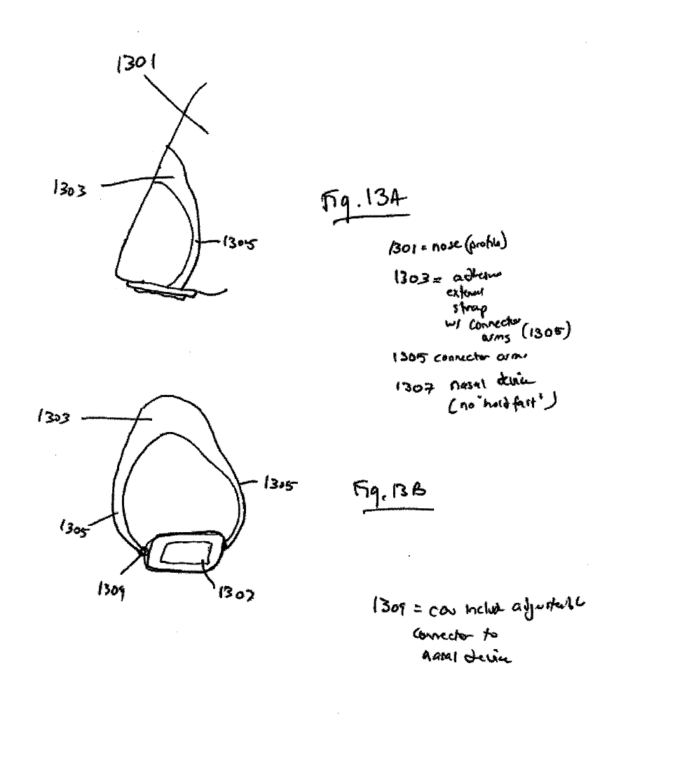

Other methods of attachment of any of these nasal devices may also be used. For example, a nasal device may be attached using a permanent or semi-permanent implant that interfaces with the device. For example, a nasal device may be anchored via a piercing, or the like. In some variations, the nasal device is an implantable device that can be implanted deep within a subject's nasal cavity. For example, an expanding implant (similar to a stent) may be expanded in place upon delivery within the subject's nasal cavity. An example of this is illustrated in FIG. 12. In this example the device is inserted via a catheter that is placed in the nostril(s). Ejecting the device allows it to expand within the nostrils. Expansion may be triggered expansion; the device can be delivered into the nostril in a closed configuration and then activated to expand.

Nasal devices may also be held in place by securing on sites that are not the nose. For example, a nasal device may also be secured to a subject via a mouthpiece. A mouthpiece may be worn in the mouth, to hold the device to the nose region. Thus, a mouthpiece may include a nasal attachment that projects up to the nose, or in front of one or both nostrils.

In some variations, the nasal device is attached to the subject's ears, around the subject's head, or to a pair of glasses.

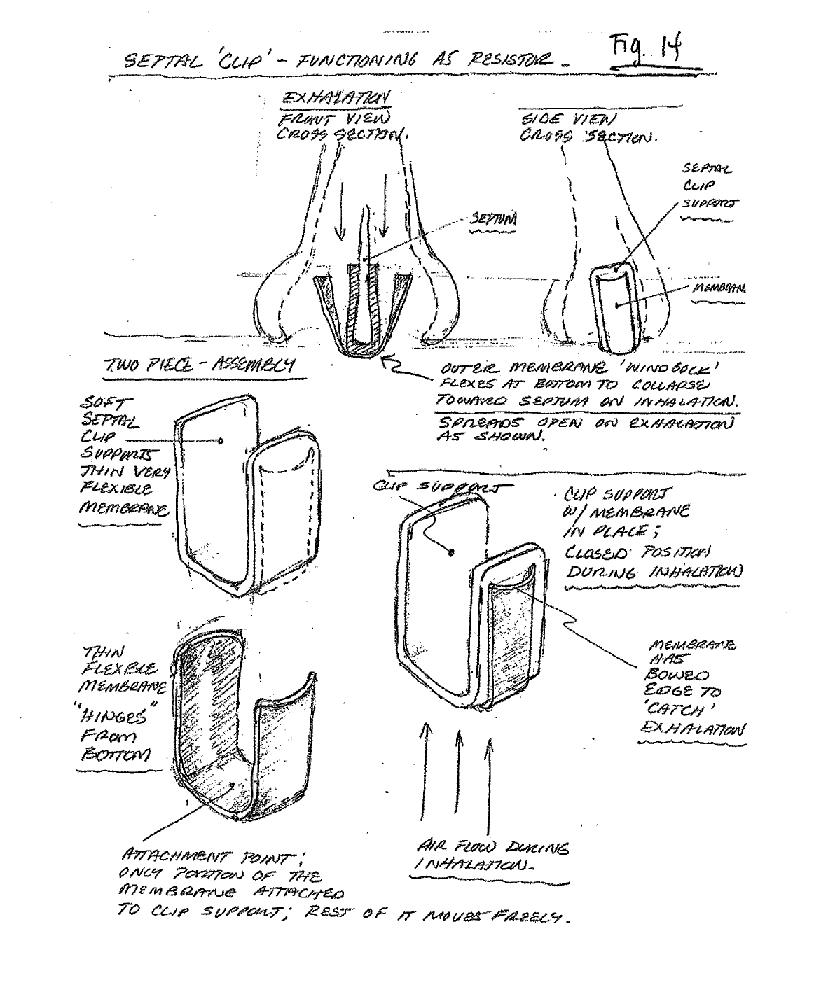

Although many of the variations previously described include adhesively attaching the device(s) to the subjects skin around the nostrils, this region may become irritated or may not well tolerate an adhesive or expandable member. In some variations, the nasal device may be configured to adhesively attach to other parts of the subject's nose or face. For example, the device may be connected to the bridge of nose, where it may be held in/over the nose. The device may thus be mechanically sealed over the nose. FIGS. 13A and 13B illustrate one variation of such a device. This variation of nasal device includes an adhesive strap that fits over the bridge of the nose, and has two connector arms that connect to either side of a nasal device. The nasal device is held against the nostril openings. In some variations the strap and/or connector arms may be elastic or slightly elastic so that the region of the device including the airflow resistor may be pulled upwards to mechanically seal with the subject's nostrils. The device may partially project into the nostrils. Similarly, in some variations, the device may be anchored off, or out, of the subject's mouth and may push up to seal against the nostril openings.

In some variations, the nasal devices described herein do not include a central opening or passageway. For example, a nasal device may be U-shaped, or may use all or a portion of the nose itself to limit airflow or increase expiratory resistance. One example of this was given above in FIGS. 4A-4C. FIG. 14 illustrates and describes another variation.

In FIG. 14, the nasal device is inserted into the nose and attached to the septum (the septal “clip” portion). Similar to FIG. 4A-4C, this device also controls the orifice size of the nostrils during expiratory flow. In FIG. 14, however, the device includes a membrane or member that expands from the septal clip region to occlude airflow during exhalation. The membrane may form a pouch that catches and/or blocks (e.g., by partially inflating) the airflow.

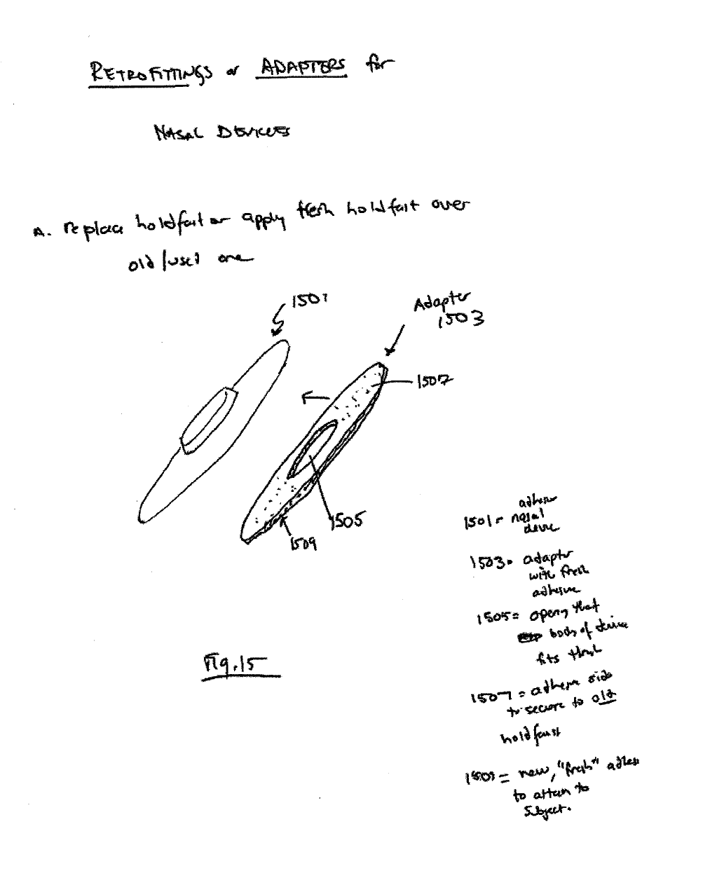

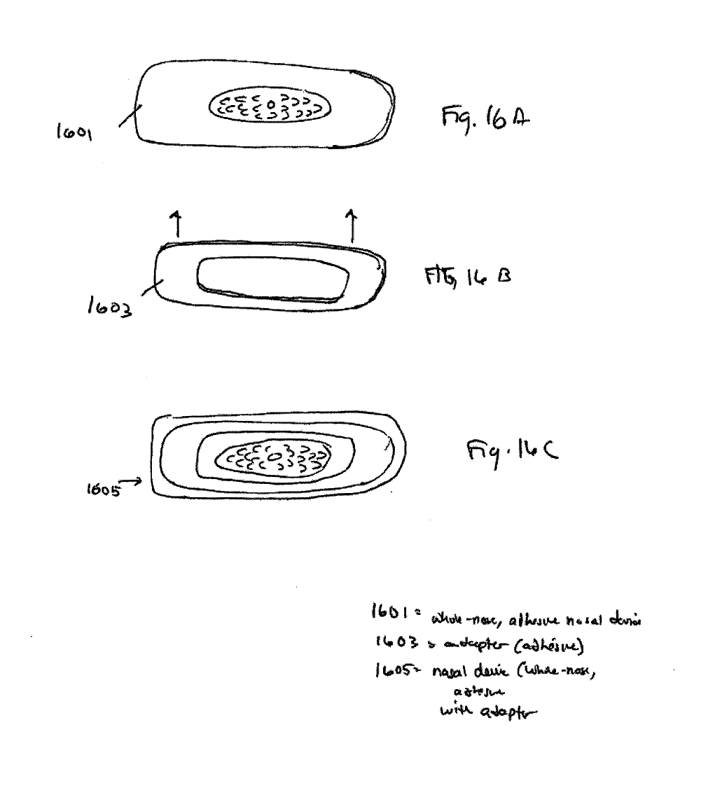

Many of the variations described are single-use nasal devices. Such nasal devices may also be adapted for multiple-use. For example, the airflow resistor region may be separable or interchangeably connected to a holdfast region. In some variations “retrofitters” or adapters may also be included or used with a nasal device, allowing the nasal device to be re-used. FIGS. 15-16C illustrate two variations of adapters that may be used.

In FIG. 15, an adhesive nasal device 1501 may be directly applied to a subject via the adhesive holdfast, and then removed. After removal, an adapter 1503, which has double-sided adhesive and an opening for the airflow resistor/passageway, may be retrofitted to the nasal device. One side of the adhesive may be attached to the nasal device (e.g., the used adhesive side), and the other side may be attached to the subject.

FIG. 16 shows whole-nose nasal device having multiple mini-flaps forming the airflow resistor region in the center. The adhesive holdfast surrounds the resistor opening. An adapter is shown in FIG. 16B. FIG. 16C shows an assembled, retrofitted nasal device including the adhesive adapter.

Part II: Nasal Devices with Intranasal Pressure Detection

In general, a nasal device configured to sense intranasal pressure may include one or more pressure sensors passing through the nasal device so that a least a portion of the pressure sensor is exposed to the intranasal region when the nasal device is worn. Typically, a nasal device has a top side or region that faces “in” (e.g., exposed to the intranasal space) when the device is worn, and a bottom side or region that faces “out” (e.g., away from the intranasal space) when the device is worn. The pressure sensor may include (or be connected to) a pressure transducer that detects the pressure felt by the sensor and converts it to a signal for measurement, storage and/or display.

In some variations, the intranasal pressure sensors included as part of these devices and systems includes a cannula. The cannula may be configured as a hypotube, and typically includes a central lumen and a distal opening. The distal opening is configured to be placed in fluid communication with the intranasal space so that the lumen of the cannula reflects the intranasal pressure. The cannula may extend through the nasal device and eventually connect to a pressure transducer. For example, the cannula may connect to the pressure transducer via tubing, permitting an external pressure transducer to be used, including a re-usable pressure transducer. In other variations, the pressure transducer is formed as part of the pressure sensor, and may be directly connected to the nasal device.

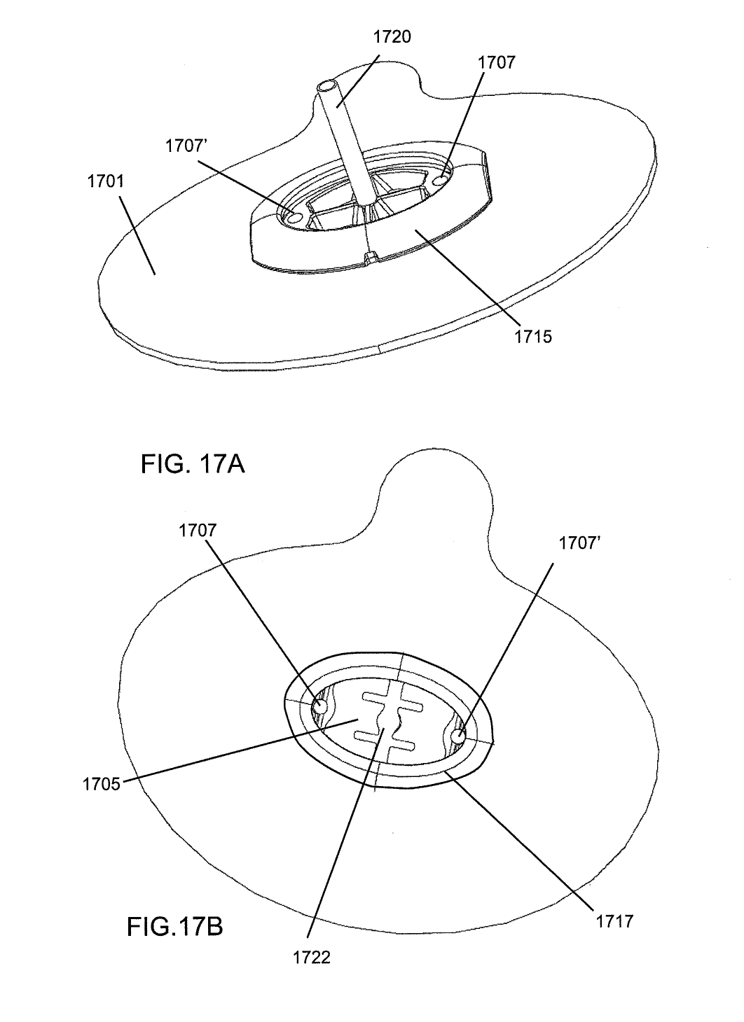

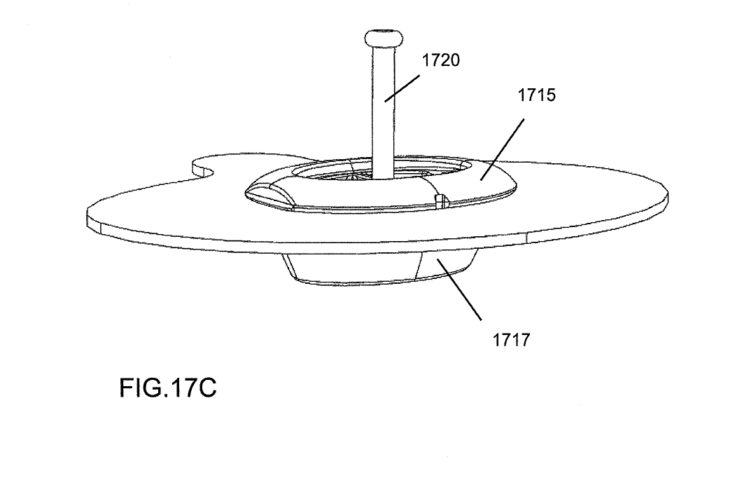

FIGS. 17A and 17B illustrate one variation of a nasal device including a pressure sensor that is a cannula. The nasal device shown in FIGS. 17A-17B, and many of the subsequent figures, is only one variation of a nasal device which may form part of an intranasal pressure-sensing nasal device. Other variations, including layered or substantially flat nasal devices, whole-nose nasal devices, entirely flexible, semi-flexible, and rigid nasal devices are illustrated in the patent applications incorporated by reference above, and may be adapted to sense intranasal pressure as described herein.

In FIG. 17A, the nasal device includes a flexible, adhesive holdfast region 1701 that surrounds a rim body region 1715. A flap valve 1705 (visible in FIG. 17B) is contained within the rim body. In this variation, the rim body is formed of an inner portion 1717 (visible in FIG. 17B) and an outer portion 1715 (visible in FIG. 17A), and a central passageway through the rim body is valved by the flap valve 1705, so that the flap valve opens during inhalation, permitting inhalation through the device with only nominal resistance, but closing to inhibit exhalation through the device. During exhalation, airflow through the device may be limited to one or more “leak pathways” formed through the device. Leak pathways may be formed through any portion of the nasal device, including the body (e.g., rim body), the holdfast, or the airflow resistor. In FIGS. 17A and 17B, the leak pathways 1707. 1707′are formed through the rim body adjacent to the flap valve 1705.

A cannula 1720 passes through the airflow resistor of the nasal device shown in FIGS. 17A and 17B. In this embodiment the cannula is the intranasal pressure sensor, and is secured through the nasal device so that the proximal end of the cannula extends from the bottom side of the device (visible in FIG. 17A). The distal end of the cannula is not visible in FIGS. 17A-17B, but is located behind the crossbeam 1722 in FIG. 17B. This distal end includes an opening into the central lumen of the cannula, and this opening is exposed to the pressure within the intranasal space when the nasal device is applied to a subject. The proximal end of the cannula extends from the nasal device (from the bottom of the nasal device, away from the subject's nose) when the nasal device is worn by a subject, and may be connected to a pressure transducer.

In any of the variations described herein, the intranasal pressure sensor may be positioned relative to the nasal device so that it is exposed to the intranasal pressure when the device is worn, but is relatively isolated from the flow through the device. For example, when a cannula is used to detect intranasal pressure, the cannula may be positioned to sense a pressure signal reflecting the actual intranasal pressure, rather than an signal representing both pressure and flow. Thus, the pressure sensor or cannula opening may be positioned to get an accurate pressure signal (rather than a “pressure+flow” signal). In FIGS. 17A-17C, the cannula opening is protected from flow through the device by placing the cannula opening behind structures in the nasal device. In some variations, the pressure sensor or cannula may be protected from the effect of flow by placing the sensor or cannula opening out of the flow path. In some variations, the end of the cannula may be pointed in the direction of flow through the device, but the opening to the cannula is positioned on a side or face of the cannula.

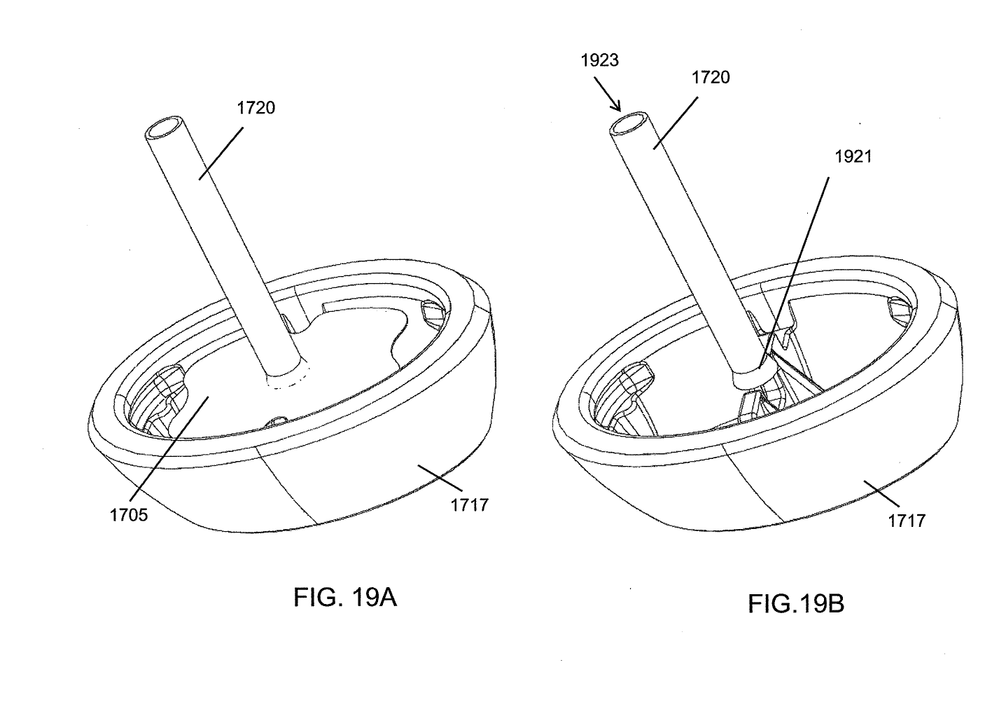

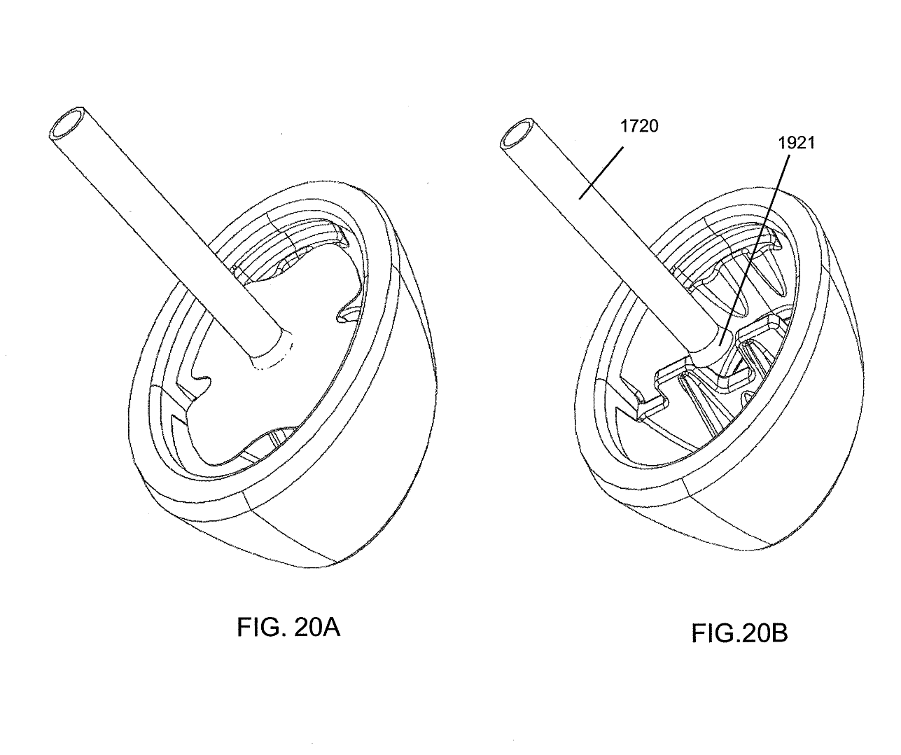

The cannula in FIG. 17A-17C passes through the flap valve, and may help secure the flap valve within the nasal device, as described in greater detail below for FIGS. 19A-20B.

FIG. 17C shows a side perspective view of the nasal device including an intranasal cannula. In FIG. 17C, the device is oriented so that the side of the device configured to face the intranasal space 1717 is down, and the side that faces away from the intranasal space 1715 is up. The intranasal pressure sensor (cannula 1720) extends from the side configured to face away from the intranasal space 1715. The cannula may be connected to tubing or directly to a pressure transducer (not shown).

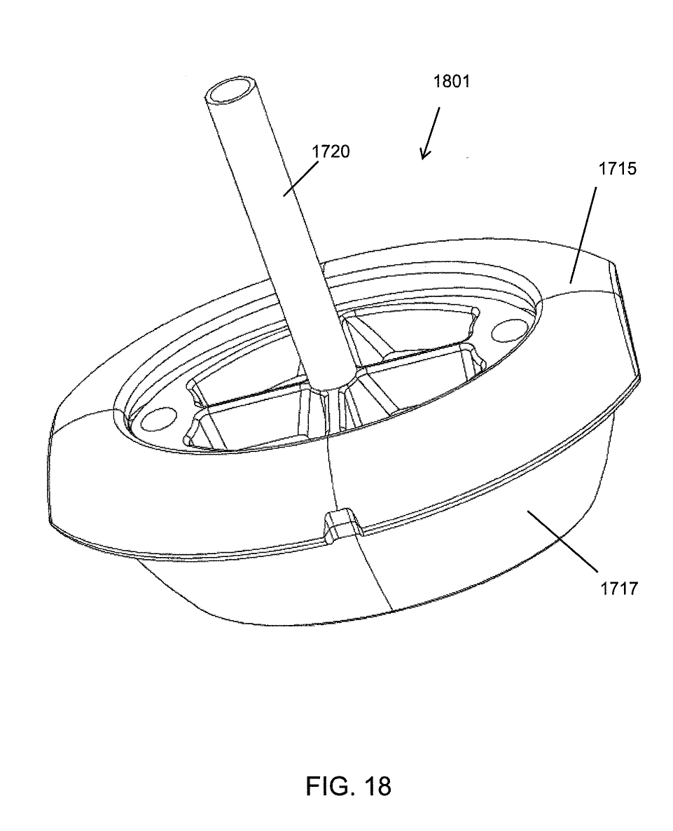

FIG. 18 illustrates greater detail of the body portion of the nasal device shown in FIGS. 17A-17C. The adhesive holdfast is not shown in FIG. 18. In FIG. 18 it is apparent that the cannula 1720 is attached to the body of the nasal device 1801, and passes through the nasal device. FIGS. 19A and 19B illustrate the body 1801 of FIG. 18 with the upper region 1715 removed.

FIG. 19A illustrates the flap valve 1705 spanning the opening or central passageway through the nasal device. The intranasal pressure cannula 1720 passes through the flap valve in this example, and may help secure the flap valve centered in position within the passageway. The airflow resistor in this variation is configured so that the flap valve opens during inhalation by bending “down” in FIG. 19A into the lower rim body 1717 region of the rim body. The upper rim body region (not shown) is configured to limit the movement of the flap valve so that it cannot bend “up” during exhalation.

In FIG. 20B, the flap valve 1705 shown in FIG. 19A has been removed so that the distal end 1921 of the cannula 1720 is visible. The cannula distal end 1921 in this example is flanged or flared so that the flap valve cannot pass over the distal end as it moves during respiration. The flanged distal end may also help secure the cannula within the nasal device. In some variations, the distal end is not flanged, but includes a lip or other region (e.g., protrusion, etc.) to prevent the flap valve from passing over the distal end. The distal end 1921 of the cannula 1720 is opened to expose the lumen 1923 of the cannula (not visible).

The open distal end of the cannula is exposed to the intranasal side of the device, which is the side of the device that is continuous with the nasal passage when the device is worn by a subject, even when the airflow resistor is closed. In this example, the distal end of the cannula is open to the intranasal side of the nasal device, although a portion of the rim body 1717 is opposite from the opening. As mentioned, this configuration may isolate the cannula from the effect of flow (e.g., dynamic pressure) so that the static component of pressure is more reliably measured. This static component may reflect the pressure seen by the walls of the nasal cavity. This may be more readily apparent in FIGS. 20A and 20B, which are rotated view of FIGS. 19A and 19B, respectively. In FIG. 20B, the separation between the distal end 1921 opening of the cannula 1720 is more readily apparent. This configuration may also protect the opening of the cannula lumen from contamination, while still allowing the cannula lumen to sense the intranasal pressure.

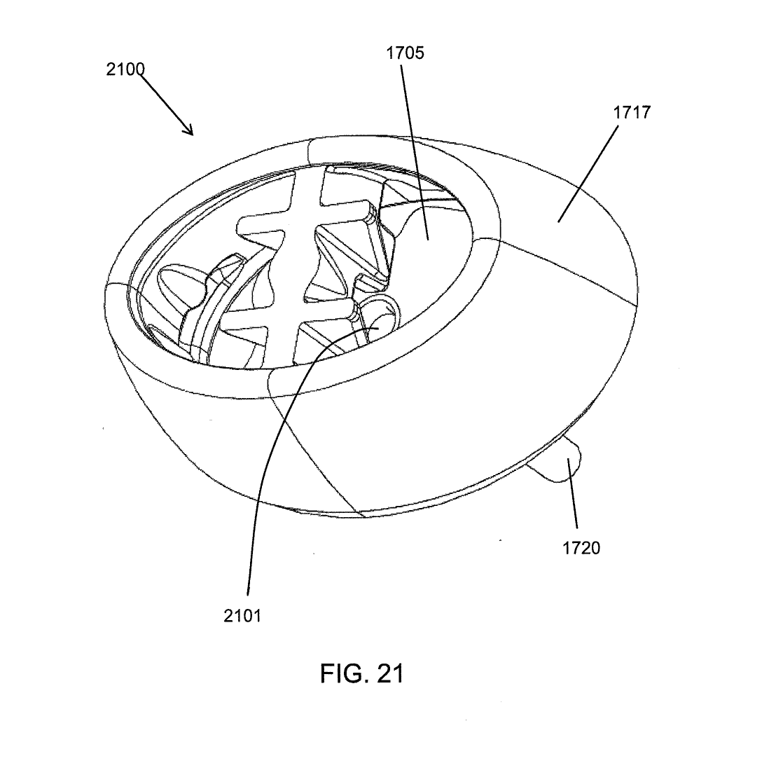

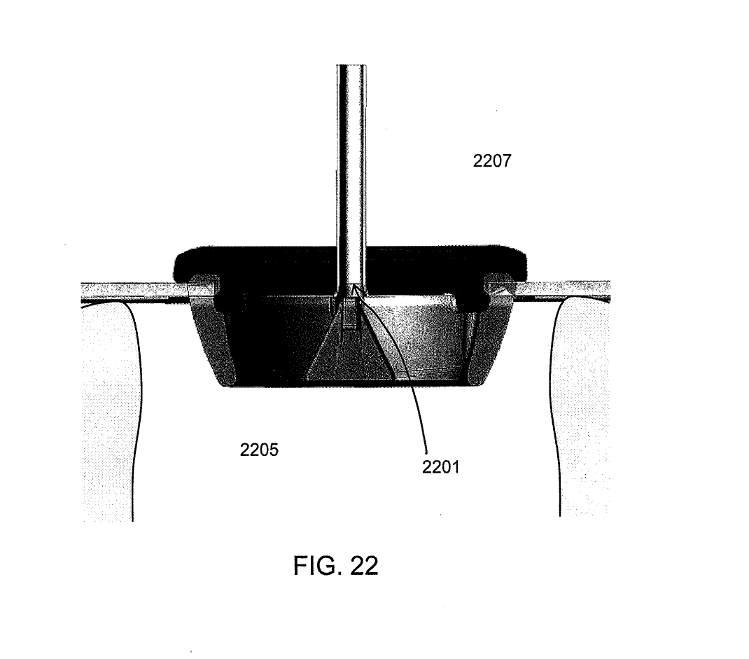

FIG. 21 shows a perspective view of the face of the nasal device rim body that is configured to be placed within the subject's nasal cavity. Thus, FIG. 21 is a rotated view of the device shown in FIGS. 19A-20B. In FIG. 21, the opening through the nasal device 2100 is continuous with the opening 2101 into the cannula 1720 even when the airflow resistor (e.g., flap valve 1705) is closed, as shown here. The continuity of the cannula and the intranasal space 705 is even more apparent in the cross-section through the device shown in FIG. 22, as indicated by the arrow 2201. In this example, the nasal device is applied over a subject's nostril, so that the airflow resistor can inhibit exhalation through the nostril more than inhalation through the nostril, resulting in a difference in intranasal pressure 2205 compared to pressure outside of the nasal cavity 2205.

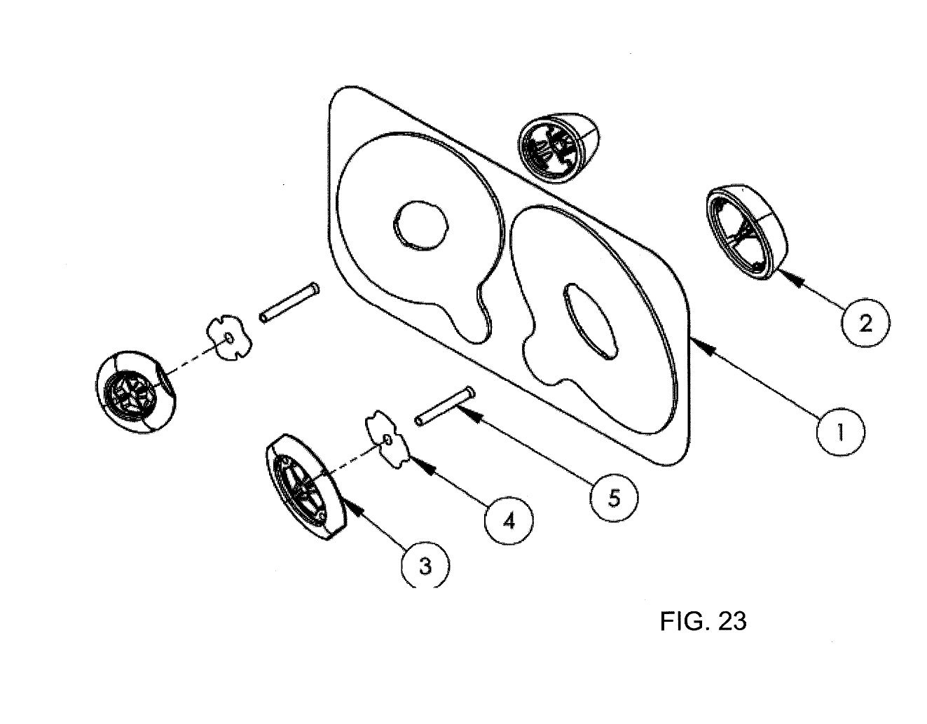

FIG. 23 shows an exploded view of two nasal respiratory devices configured for monitoring intranasal pressure. The nasal devices described herein may be assembled in any appropriate manner. In some variations the cannula 5 is secured within the nasal device so that it is secured within one portion of the rim body, and may help secure or position the airflow resistor. For example, in FIG. 23, the inner rim body region 2 may be first secured to the adhesive holdfast (shown on backing 1) region. The cannula 5 may then be passed through the flap valve 4, so that the flap valve is positioned slightly proximal of the distal end of the cannula (the flared end in this variation). The cannula is then passed through the outer body region 3 and this sub-assembly including the cannula, flap valve and outer body region can then be secured to the inner body region 2 and the adhesive holdfast. The cannula may be secured to the outer body region by an adhesive (e.g., glue, cement, etc.) or by connecting tubing to the cannula from the outer face of the device. Other methods of assembly may be used for this variation.

In operation, the intranasal pressure-sensing nasal devices may be worn by a subject to monitor intranasal pressure as part of a treatment. For example, a nasal device having an airflow resistor that inhibits exhalation more than inhalation may be used to treat one or more respiratory disorders, including sleeping disorder such as snoring or sleep apnea. Thus, the device may be applied to a patient so that nasal breathing, particularly during exhalation, is limited by the device. Once applied, the subject's intranasal pressure may be monitored using the device, which may help monitor treatment. Intranasal pressure may be monitored to refine the device (e.g., the resistances applied by the device), or to confirm the fitness (or fit) of the nasal device. Alternatively, the intranasal pressure measurements may provide information about the individual user's physiology or physiologic response which will help predict clinical effectiveness, or help guide which device resistance is optimal or suitable. In some variations, the intranasal pressure may be monitored to further diagnose the subject.

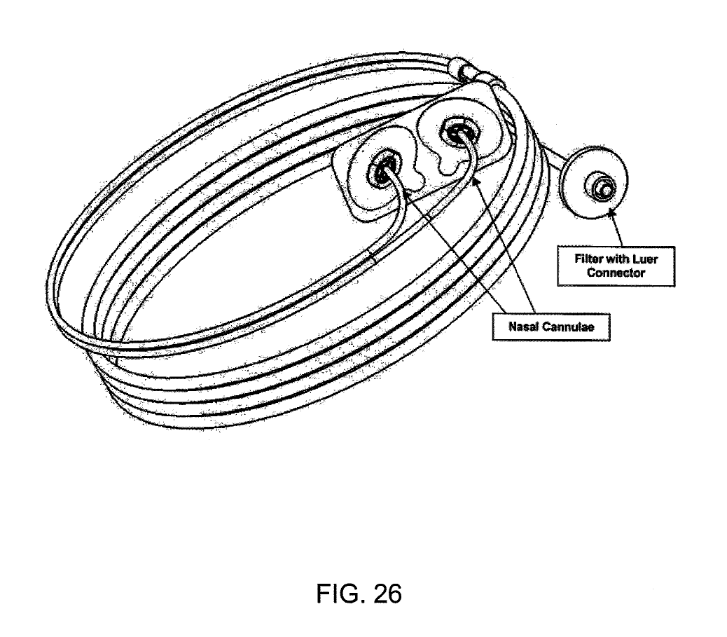

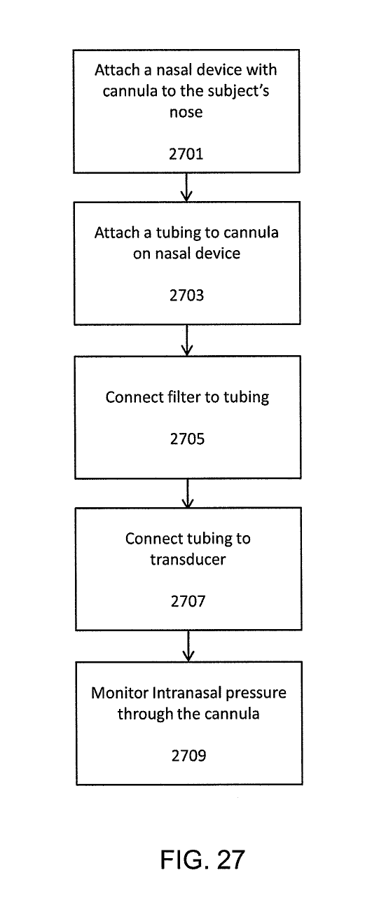

For example, in variations in which a nasal device including an intranasal pressure sensor configured as a cannula is applied to treat a subject, the cannula may be connected to a pressure transducer. For example, FIG. 27 outlines one method of using the device. In the first step, a nasal device with a cannula (or hypotube) passing through the nasal device is secured to the subject's nose 2701. For example, two disposable nasal devices (such as those shown in FIGS. 17-21) may be adhesively secured to the subject's nostrils. Each nasal device is placed just inside a nostril and is held in place by the adhesive holdfast. Exhalation through the device is limited to the leak pathways, which increases the airway pressure during the therapy. This expiratory resistance may help maintain the patency of the airway. Tubing may then be attached to the cannula projecting from of each nasal device 2703 in order to connect the sensor to a pressure transducer. Optionally, the tubing may include or may be connected to a filter 2705 before connecting to a pressure transducer 2707.

Any appropriate pressure transducer may be used. In general, a pressure transducer is a transducer that converts pressure into an electrical signal. Although there are various types of pressure transducers, one of the most common is the strain-gage base transducer. The conversion of pressure into an electrical signal may be achieved by the physical deformation of strain gages or diaphragm of the pressure transducer. Circuitry such as a wheatstone bridge may be used to determine deflection of the transducer and current/voltage changes. For example, pressure applied to the pressure transducer produces a deflection of a diaphragm which introduces strain that will produce an electrical resistance change proportional to the pressure. The system may also include one or more components for monitoring, measuring or recording the intranasal pressure, typically by receiving the output of the pressure transducer. Monitoring of the intranasal pressure may be performed as part of (or in addition to) other polysomnogram (PSG) measurements.

For example, the intranasal pressure may be monitored through the hypotube by connecting the output of the pressure transducer to a module (e.g., a computer, etc.) for recording, analyzing and/or displaying the intranasal pressure 2709. This intranasal pressure monitoring may be performed by dedicated machinery (a monitoring unit) or a versatile monitor, including a computer with computer software/hardware. The pressure may be recorded and/or displayed. For example, the data may be digitized and stored digitally or displayed.

When the nasal devices including the intranasal pressure monitoring are used, the subject may be instructed to breathe normally through their mouth. In particular, when the device is first applied over the subject's nostrils, mouth breathing may help the subject acclimate to the nasal devices. Thus, the subject may fall asleep wearing the nasal devices.

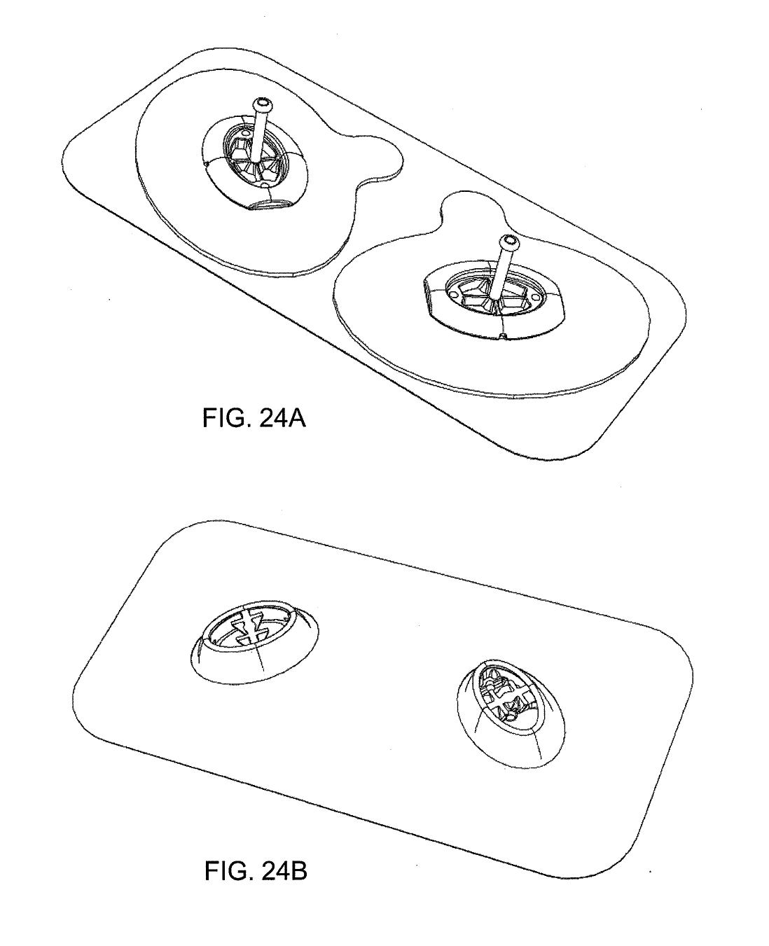

Thus, a system may include one or more nasal respiratory device adapted for measuring intranasal pressure as described herein. For example, two nasal respiratory device adapted for measuring intranasal pressure may be included in variations having a nasal device for each nostril. FIG. 24A-24B illustrates one variation of a portion of a system including two nasal respiratory devices that are packaged together. These nasal devices are disposable, although re-usable devices are also contemplated.

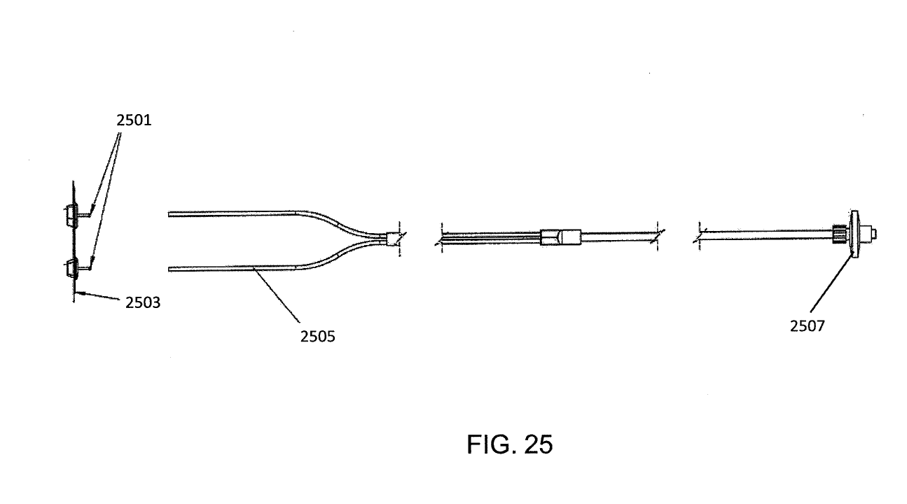

Systems for monitoring intranasal pressure may also include one or more pressure transducer(s) configured to convert the intranasal pressure to an electrical signal for analysis, storage and/or display. For example, a pressure transducer for each nostril may be included as part of the system. In variations in which the sensor attached to the nasal device is configured as a cannula, the system may also include one or more sets of tubing configured to connect the pressure sensor (cannula) to the transducer(s). For example, FIG. 25 illustrates a system including a pair of nasal devices 2501, each of which include a cannula, a set of tubing 2505 that is configured to connect the lumen of each cannula a pressure transducer (not shown), and a filter 2507. In this example, the tubing from each separate nasal device may be merged so that the pressure from each intranasal region is combined and a single intranasal pressure is seen by the transducer. The transducer and any pressure measurement, analysis and/or display system may also be included as part of a system for monitoring intranasal pressure.

FIG. 26 illustrates another variation of a system for measuring intranasal pressure, similar to that shown in FIG. 25. In FIG. 26, the system includes a pair of nasal devices each including an intranasal pressure sensor configured as a cannula that passes at least partially through the nasal devices, and a set of tubing. In this variation, the tubing is shown already connected to the cannula of the nasal device. Thus, the system may include the tubing pre-attached, or it may be attached separately. In some variations, the tubing is re-usable. In some variations, the tubing is permanently affixed (or bonded) to the nasal device(s).

### Part III: Billowing Valves

Passive nasal respiratory devices are configured to be placed in communication with a subject's nasal cavity so that airflow through the subject's nose can be regulated to inhibit exhalation more than inhalation. In general, a passive nasal respiratory device includes one or more airflow resistors that are configured to achieve this. In particular, passive nasal respiratory devices may include an airflow resistor configured as a billowing airflow resistor.

As used herein, a billowing airflow resistor includes two or more layers that are adjacent to each other and contact each other during exhalation, but become slightly separated from each other during inhalation, so that air can pass between the two layers. The first layer and the second layer are separated but substantially parallel as the first layer billows apart from the second layer during inhalation. This billowing reduces the resistance to inhalation through the device. A billowing airflow resistor may also be referred to as a “pillowing” airflow resistor; pillowing may describe the appearance of the first layer relative to the second layer when the two layers separate during inhalation.

For example, the layers may be configured so that during exhalation the layers press against each other (and may at least partially seal) to restrict airflow through the device. For example, during exhalation the first and second layer may lie adjacent to each other so that at least a portion of the two layers overlap. Thus, the layers may be positioned adjacent to each other so the edge regions of the layers overlap. The second layer may therefore be referred to as a seal or seat layer because the first layer can seal against it during exhalation, preventing or liming airflow. The second layer may be a rigid layer that supports the more flexible first layer. Alternatively, in some variations the first and second layers are adjacent to each other in a side-by-side configuration. During exhalation the second layer and the first layer sit next to each other (e.g., on a support layer), and during inhalation the flexible first layer separates from the second layer to permit airflow.

Billowing airflow resistors are distinguishable from flap-valve type airflow resistors because the surfaces forming the billowing airflow resistor typically separate slightly but remain substantially parallel during inhalation, rather than opening as a flap in which the flap surface may open away from the plane of the closed configuration. A billowing airflow resistor may be constrained so that the flexible or moveable region does not swing open at an angle from the plane of the closed configuration. For example, the edge region of the first layer stays substantially parallel to the second layer when the airflow resistor is open during inhalation. This is described in greater detail below.

The passive nasal respiratory devices described herein may generally be referred to as “nasal devices” or “nasal respiratory devices”. Many of these nasal devices are adhesive nasal devices. An adhesive nasal respiratory device is one variation of a general nasal respiratory device in which an adhesive holdfast region is used to secure the device in fluid communication with one or both of a subject's nostrils.

A nasal respiratory device, including an adhesive respiratory device, may be used to regulate a subject's respiration. For example, the device may create positive end expiratory pressure (“PEEP”) or expiratory positive airway pressure (“EPAP”) during respiration in a subject wearing the device. The adhesive respiratory devices and methods described herein may be useful to treat a variety of medical conditions, and may also be useful for non-therapeutic purposes. The devices and methods described herein are not limited to the particular embodiments described. Variations of the particular embodiments described may be made and still fall within the scope of the disclosure. Examples and particular embodiments described are not intended to be limiting.

As used herein, a nasal device may be configured to fit across, partly across, at least partly within, in, over and/or around a single nostril (e.g., a “single-nostril nasal device”), or across, in, over and/or around both nostrils (“whole-nose nasal device”). Both single-nostril nasal devices and whole-nose nasal devices may be referred to herein as “adhesive nasal devices,” and (unless the context indicates otherwise), any of the features described for single-nostril nasal devices may be used with whole-nose nasal devices, and vice-versa. In some variations, an adhesive nasal device is formed from two single-nostril nasal devices that are connected to form a unitary adhesive nasal device that can be applied to the subject's nose. Single-nostril nasal devices may be connected by a bridge (or bridge region, which may also be referred to as a connector). The bridge may be movable (e.g., flexible), so that the adhesive nasal device may be adjusted to fit a variety of physiognomies. The bridge may be integral to the nasal devices. In some variations, single-nostril nasal devices are used that are not connected by a bridge, but each include an adhesive region, so that (when worn by a user) the adhesive holdfast regions may overlap on the subject's nose.

Layered nasal devices are of particular interest, and are described more fully below. Layered adhesive nasal devices may include two or more layers. For example, a layered nasal device may include an adhesive holdfast layer and an airflow resistor layer. These layers may be composed of separate layers, and these layers may be separated by other layers, or they may be adjacent. The adhesive holdfast layer may be itself formed of layers (optionally: a substrate layer, a protective covering layer, an adhesive layer, etc), and thus may be referred to as a layered adhesive holdfast. Similarly, the billowing airflow resistor may be formed of multiple layers (optionally: a flexible layer, a seal face layer, etc.), and thus may be referred to as a layered billowing airflow resistor. In some variations, the layered adhesive holdfast and the layered billowing airflow resistor share one or more layers. For example, the flexible layer and the adhesive substrate layer may be the same layer, in which opening or cuts in the flexible layer of the airflow resistor are cut from the substrate layer material of the adhesive holdfast. As used herein, a “layer” may be generally planar geometry (e.g., flat), although it may have a thickness, which may be uniform or non-uniform in section.

As used in this specification, the singular forms “a,” “an,” and “the” include plural reference unless the context clearly dictates otherwise. The following descriptions including various design parameters or goals, and methods and devices which fit the design parameters or goals. The devices and methods described herein (and recited by any claims) are not limited to any particular theory of operation.

In general, the nasal devices described herein include a holdfast and at least one billowing airflow resistor. As will be apparent from the figures, many of these devices may be applied and removed to/from the subject's nose by user without special tools. In some variations, a subject may use an applicator to apply the device (e.g., to help align it).

In operation, a nasal device is placed in communication with one or both of a subject's nostrils to modify the flow of air through the subject's nose. Thus, the nasal devices described herein include one or more airflow resistors for modifying the flow of air through the nose in at least one direction. As mentioned, the billowing airflow resistors described herein are configured to occlude airflow through the device (e.g., an opening or openings in the device) in one direction more than it occludes airflow in the opposite direction. In particular, the billowing airflow resistors are generally configured to inhibit airflow during exhalation through the device more than inhalation through the device. Resistance to inhalation may be increased minimally, negligibly, or not at all.

Any of the nasal devices described herein may also include one or more leak pathways through which air can pass when the valve is otherwise closed. The leak pathway may be separate from the billowing airflow resistor, or it may be part of the airflow resistor (e.g., passing through an opening in both the flexible and sealing face layers of the airflow resistor that overlap even during exhalation). A leak pathway may pass through any appropriate region of the device, including the holdfast region.

A nasal device may be configured to treat snoring, or any other sleep disordered breathing, as described briefly above. For example, a subject may apply a respiratory device over his nose (one or both nostrils) by exposing an adhesive on the holdfast of the device (e.g., by removing a protective cover material from an adhesive region of the holdfast) and applying gentle pressure to adhere the device around the nostrils. In this way, the device may be seated around the nasal orifice (and may project at least partly into the nostrils) and form at least a partial seal between the nostrils and the device so that the majority of flow into and out of the nostrils passes through the passageways of the nasal device. Once the device is applied to the subject's nose, respiration through the nostrils may be regulated. During inhalation in a subject wearing such a device, the subject may breathe through the nose (and thus through the nasal device). During exhalation, the nasal device provides greater resistance to airflow through the device. Resistance to exhalation may be limited (or set) by a leak pathway. A subject may still breathe predominantly though the nose (and the nasal device) during exhalation, but may also breathe at least partly through the mouth.

It may also be beneficial for a subject to wear a nasal respiratory device over an extended period of time (e.g., during a period of sleep). Described below are variations of nasal devices (including adhesive nasal devices) that may be comfortably worn and secured in or over the subject's nose or nasal passages. In some variations, a grip (e.g., a tab, handle, strap, or other additional interface region) may be included to help secure the device to the subject's nostril, nose or face, and may additionally or alternatively be helpful in positioning or manipulating (e.g., gripping) the device, particularly when it is being applied. This additional interface region may be formed of the same material as the holdfast region.

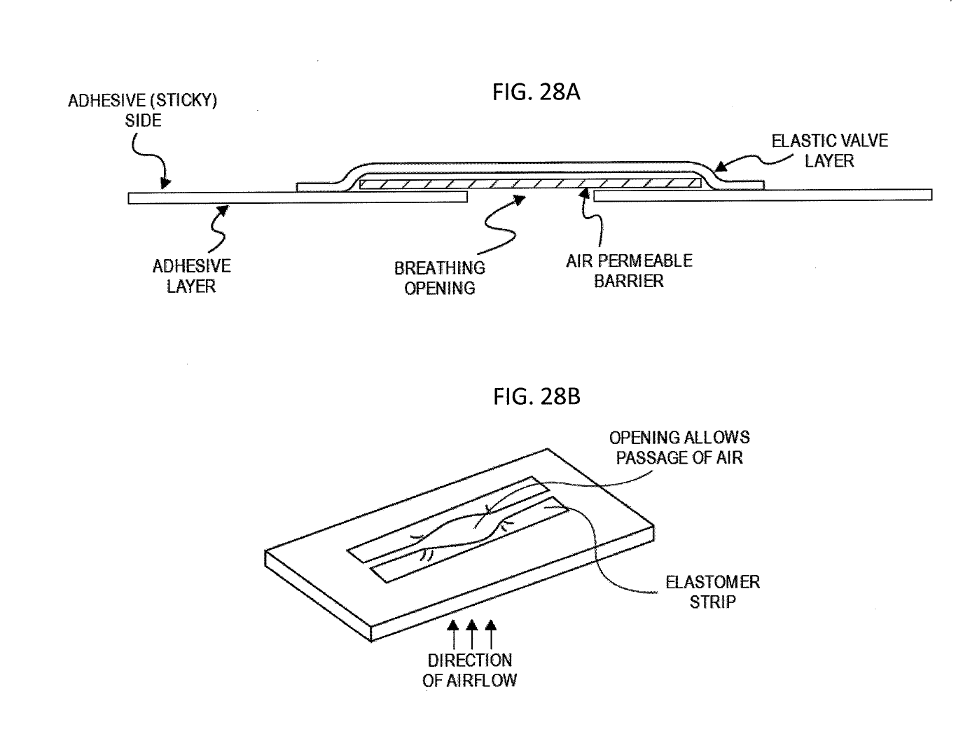

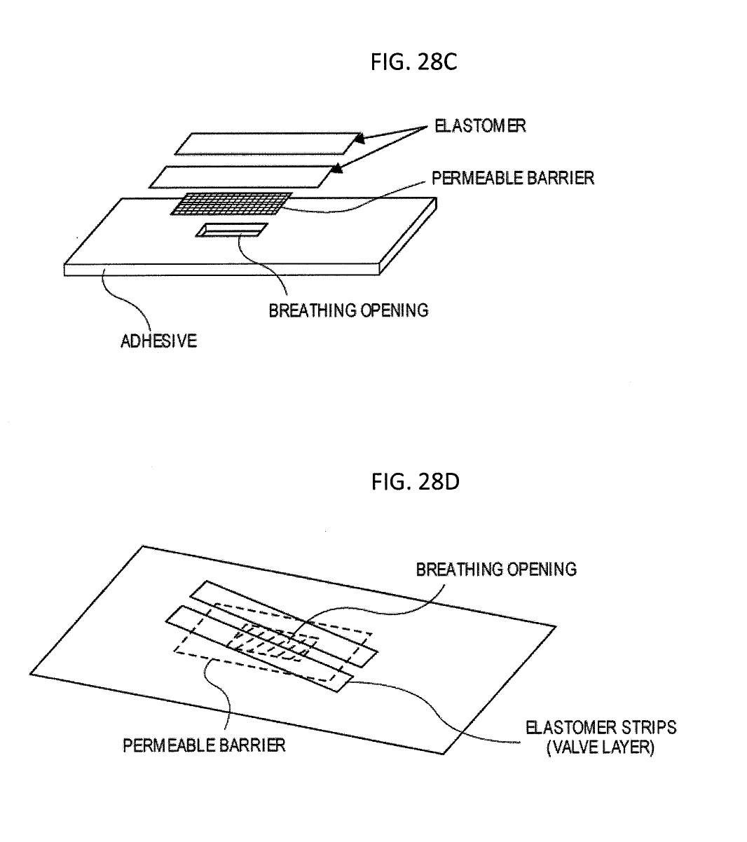

FIGS. 28A-28D show one variation of a nasal device including a billowing airflow resistor. This device is a multi-layered device. The device shown has three layers: an adhesive (adhesive holdfast) layer, a flexible layer (the elastomeric layer), and a second layer that is less flexible, and is positioned adjacent the flexible layer. This second layer is air permeable. For example, the second layer may be a mesh or otherwise perforated layer. These layers are labeled in FIG. 28A. FIGS. 28A and 28B show the arrangement of these layers to form one variation of an adhesive nasal device. The adhesive layer forms a substrate onto which the airflow resistor layers (the first and second layers) are secured. An opening through the holdfast is formed, the layers forming the airflow resistor extend across the opening. For example, the opening through the adhesive holdfast is covered with second (air-permeable layer), such as a mesh. The edges of this layer may be secured to the adhesive layer by any appropriate method (including using the adhesive of the adhesive layer, or an additional adhesive). Two parallel strips of elastomeric material are placed adjacent to the air-permeable layer spanning the opening, and secured at the ends (and possibly along one side), forming the first or flexible layer. A gap between these two strips forms a leak pathway. In some variations, no gap is present (reducing the leak pathway). In some variations, multiple strips may be used to form the airflow resistor.

This nasal device may be placed in communication with a subject's nostril(s) so that when the subject inhales, the flexible layer (e.g., the parallel bands) billow out, away from the second, air-permeable, layer, and allow air to flow with only minimal resistance. This is illustrated in FIG. 28B. During exhalation, the strips of forming the first, flexible, layer are limited from billowing in the opposite direction by the second layer (or limiting layer) formed from the air-permeable barrier, resulting in a resistance to exhalation, as air is only allowed to pass through one or more leak pathways, such as the space between the strips forming the first layer.

FIG. 28C shows an exploded view of the device shown in FIGS. 28A and 28B, indicating the arrangement of the layers forming the adhesive holdfast and the airflow resistor. In this example. FIG. 28D illustrates a partially transparent perspective view. As mentioned, a device such as that shown in FIGS. 28A-28D, and any of the devices described herein, may be used to treat medical conditions including snoring, and typically can be worn by a subject to provide a small resistance to exhalation (e.g., between about 0.001 and about 0.25 cm H₂O/(ml/sec), between about 0.01 and about 0.25 cm H₂O/(ml/sec), between about 0.01 and about 0.20 cm H₂O/(ml/sec), etc).

In FIGS. 28A-28D, the billowing airflow resistor is shown to include a first flexible layer and a second supporting layer. The flexible layer in this example is an elastomeric material, however, any appropriate material may be used, particularly thin, flexible materials including polyurethanes and other polymers. The second, less flexible, layer that acts as a support layer in this variation is formed of a mesh, through which air may pass when the first layer billows and separates from the second layer during inhalation. The relative flexibility or ability to stretch of any of the layers or materials described herein may be a function of the material and also a function of the arrangement of the material. For example, the first, more flexible, layer may be made of the same material as the second (less flexible) material, however the second layer may be less able to stretch or bend under nasal pressure because it is secured more tightly, supported, or otherwise stiffened.

A billowing airflow resistor is generally formed by two (or more) layers that are adjacent to each other and that separate during inhalation. In some variations, the two layers are secured atop one another. The two layers may be connected to each other, or to another region of the airflow resistor. In some variations, the edges of the first and second layers are connected along one or more edges of the perimeter. Unconnected regions may open during inhalation to allow airflow through the device.

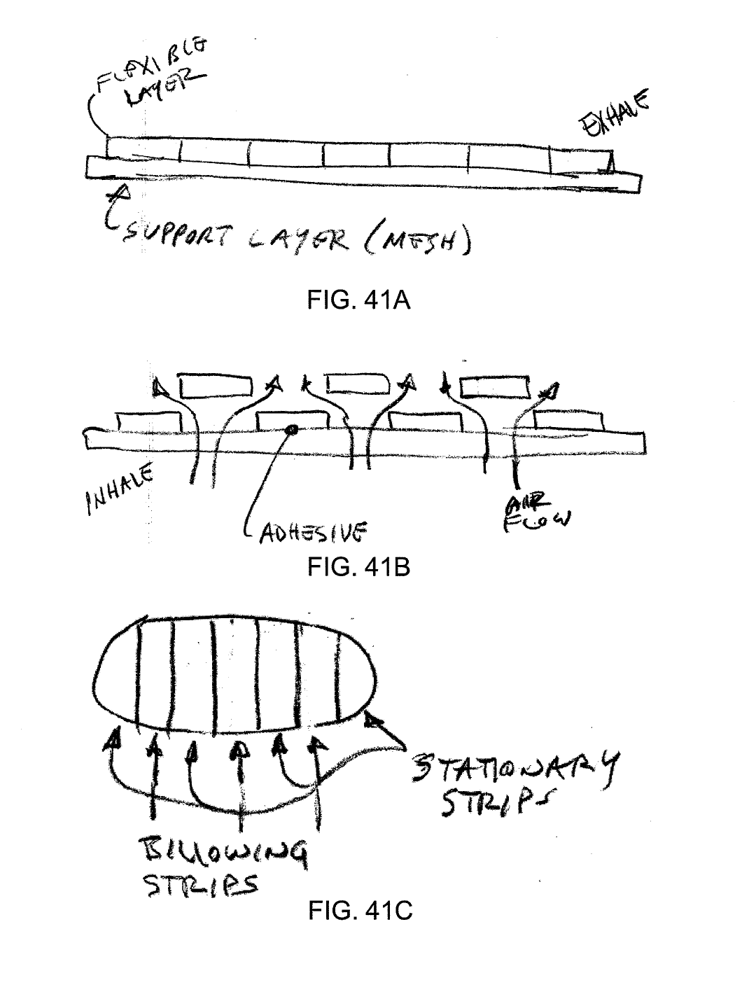

FIGS. 41A-41C illustrate another variation of a billowing airflow resistor that may be used as part of a nasal device. In FIGS. 41A-41C the flexible first layer forms both a movable and a stationary component of an airflow resistor. The second layer may be a mesh or support layer, which may be stiffer than the first layer. Portions of the first layer may are secured to the second layer (e.g., via adhesive or other method) so that non-secured portions of the first layer may billow to form openings during inhalation, as illustrated in FIG. 41B. On exhalation, the edges of the billowing portion of the first layer lie adjacent to the stationary portions of the first layer, as shown in FIG. 41A, substantially preventing expiratory airflow. In some variations a gap may be present between the stationary and the fixed portions of the first layer to act as a leak pathway, which may help control the expiratory resistance of the billowing airflow resistor. This design does not use a sealing face, as described in greater detail below, so the second layer support and stops the flexible first layer from billowing during exhalation, and may help seal the billowing strips of the first layer in plane with the stationary portions of the first layer so that an edge-to-edge seal is formed.

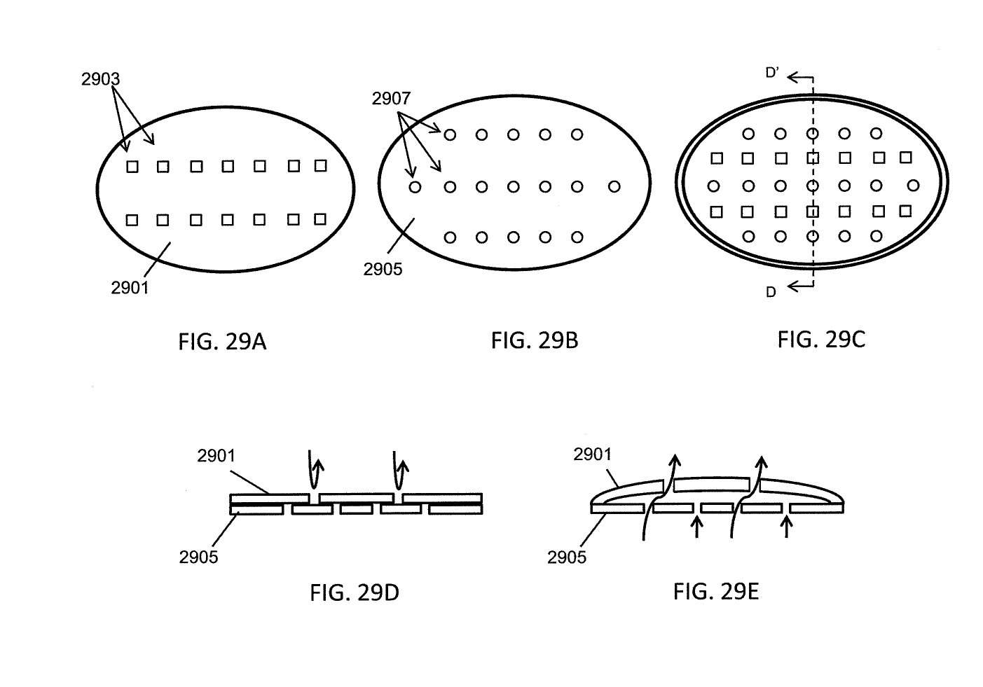

FIGS. 29A-29E illustrate one variation of a billowing airflow resistor in which the first and second layers forming the billowing airflow resistor include non-overlapping openings. In the billowing airflow resistor shown in FIG. 29A-29E the perimeter of the first and second layers are secured against each other. FIG. 29A illustrates one variation of a flexible first layer 2901, including a plurality of openings 2903. FIG. 29B illustrates a second layer 2905 that also includes a plurality of openings 2907. FIG. 29C shows the partially assembled billowing airflow resistor, in which the flexible first layer has been secured to the lower layer 2901. In this example, the perimeters of the first layer 2901 and second 2905 layer are secured together. The openings 2903 in the flexible first layer 2901 do not align with the openings 2907 of the second layer 2905, as illustrated in FIG. 29C, in which the first and second layers have been made transparent, so that the lack of registration between the openings may be seen.

At rest, the flexible first layer 2901 of the billowing airflow resistor lies adjacent to the second layer 2905. The second layer 2905 may be a limiter layer, and may be stiff (or at least less flexible than the upper layer), so that it doesn't substantially change shape at the pressures associated with inhalation and exhalation. The first layer is sufficiently flexible so that it may move, expand, and/or change shape.

FIGS. 29D and 29E show cross-sections through the billowing airflow resistor of FIG. 29C along line D-D′. FIG. 29D illustrates the operation of the billowing airflow resistor during exhalation. When a nasal device including a billowing airflow resistor such as the one shown in FIGS. 29A-29E is applied to a user, during exhalation the first and second layers may be pressed against each other, preventing the passage of air through the device, as indicated by the arrows in FIG. 29D. During inhalation, as illustrated in FIG. 29E, the flexible first layer 2901 billows away from the second layer 2905. The separation between the first layer and the second layer creates a gap between the layers, and allows air to pass through the openings 2903, 2907 in the first and second layer. During the billowing, the first and second layers remain substantially parallel to each other, although a gap permitting airflow forms between them. During inhalation, the negative pressure in the nasal cavity may draw the first layer away from the second layer, creating a space between them. For example, when the first layer is sufficiently flexible, the two layers are pulled apart during inhalation, and the flexible first layer separates from the second (sealing face layer), permitting air to flow between the two layers. This is illustrated in FIGS. 29E, 30B, 36, 40A, and 41B, described below.

The number, shape and size of passages through the first and second layers may be varied, and may help determine the resistance to inhalation and/or exhalation through the device. For the purpose of illustration in this example, the openings through the first layer are shown as square, and openings through the second layer are shown as circles, they may be any shape or size.

In some variations, a leak pathway may be formed by openings in both the first and second layer that overlap at least partially when the first and second layers are pressed against each other during exhalation. For example, in some variations, openings through the first and second layers may align with each other to from a leak pathway through the device (not shown). The shape and size of the overlapping region forming the leak pathway may be selected to choose the resistance to exhalation and/or inhalation.

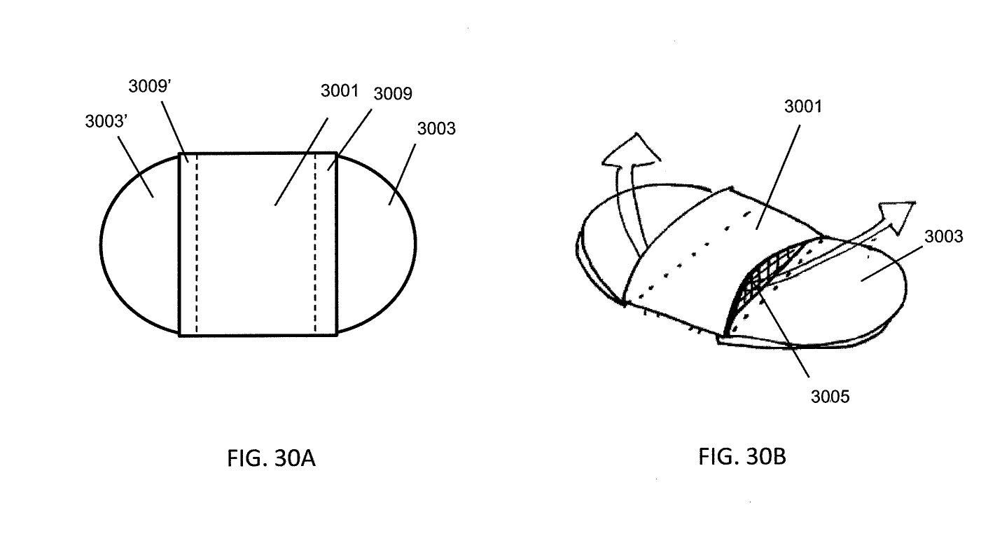

FIGS. 30A and 30B illustrate another variation of a billowing airflow resistor. In this variation, the first layer 3001 is also a flexible layer (or made of a flexible material). The second layer 3003 is adjacent to the first layer. In the top view shown in FIG. 30A, the second layer includes two (unconnected) regions 3003, 3003′ that each lie adjacent, and beneath, the first layer 3001. An additional support layer 3005 (visible in FIG. 30B) that is air-permeable (e.g., mesh) lies adjacent to and beneath the flexible first layer 3001. In this example, the support layer may be part or may be combined with the second layer 3003, 3003′.

The second layer 3003, 3003′ may also be referred to as a sealing face layer. The sealing face layer overlaps with a portion of the flexible first layer, as indicated by the dashed line region 3009, 3009′ in FIG. 30A. During exhalation, the first flexible layer is sealed against the sealing face layer 3003, 3003′, providing a relatively high resistance to exhalation through the device. FIG. 30B illustrates this billowing airflow resistor during inhalation.

In FIG. 30B, the flexible layer 3001 is drawn away from the sealing face layer 3003, 3003′ (including the support layer 3005). Air may pass through the airflow resistor, providing a much lower resistance to inhalation, as indicated by the arrows. In some variations the billowing airflow resistor includes a leak pathway, such as a hole or opening that is open during both exhalation and inhalation (or just during exhalation).

The billowing airflow resistor shown in FIGS. 29A to 30B are not shown with a holdfast. Holdfasts (and devices including holdfasts) are described in greater detail below, but a billowing airflow resistor may include any appropriate holdfast. For example, the billowing airflow resistors described herein may be included as part of a device having an adhesive holdfast that extends around the billowing airflow resistor, and secures the billowing airflow resistor to the subject's nose (e.g., nostrils) in the proper orientation, so that the flexible layer billows away from the second (e.g., sealing face) layer. This may mean that the adhesive holdfast includes an adhesive on one side, which is the same side of the device as the flexible layer.

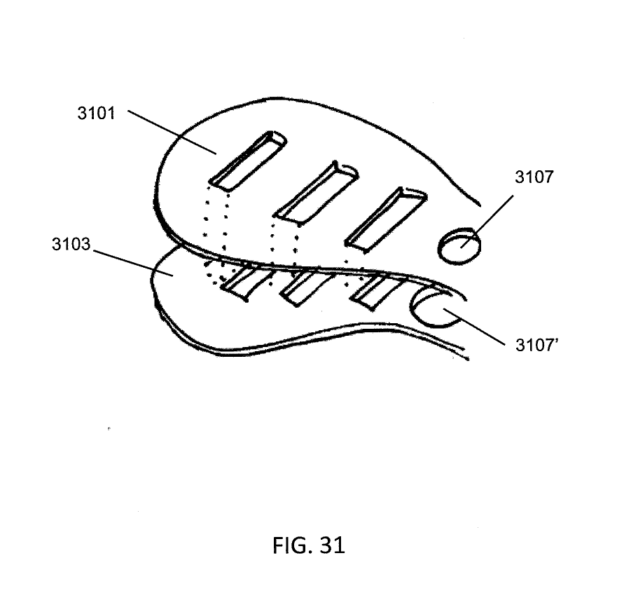

FIG. 31 shows an exploded view of another variation of a billowing airflow resistor formed by two adjacent layers. In FIG. 31, the first layer 3101 is a flexible layer that is secured adjacent to a second layer 3103. Both layers include multiple openings that are staggered or offset, so that when the first and second layer are pressed against each other, e.g., during exhalation, air cannot readily pass between them. The example shown in FIG. 31 also includes a central leak pathway 3107, 3107′ through both the first and second layers, respectively.

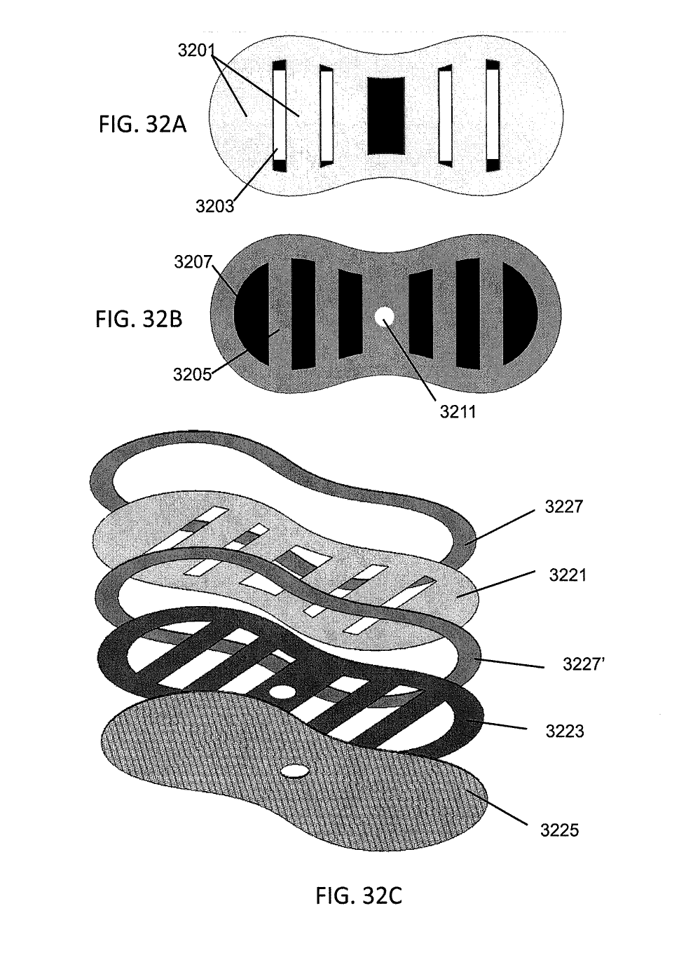

FIGS. 32A-36 all illustrate similar variations, in which the billowing airflow resistor is formed by first and second layers (flexible layers and sealing face layers, respectively) that are adjacent to each other. In these examples, the first layer is a flexible layer that includes strips that are secured across an opening through the device. The strips forming the first layer are separated by openings. For example, FIG. 32A shows a flexible layer that includes six strips 3201 (including two end regions). The strips are secured across an opening through the device, and each strip has at least one unsecured edge. In FIG. 32A, each strip is separated from adjacent strips by an opening 3203. FIG. 32B shows a sealing face layer that may be used with the flexible layer shown in FIG. 32A. In FIG. 32B, the sealing face layer is a surface against which the flexible layer can seal during exhalation, when the flexible layer is pushed against the sealing face layer. The sealing face layer includes regions 3205 that overlap with the edges of the strips forming the flexible layer. These regions may also surround openings 3207 that would be covered by the flexible layer during exhalation. A central leak path 3211 is also included through the sealing face layer.

FIG. 32C shows an exploded view of the billowing airflow resistor variation including the flexible layer 3221 and sealing face layer 3223 shown in FIGS. 32A and 32B, respectively. In FIG. 32C, the billowing airflow resistor also includes a mesh layer 3225, which is a support layer that, in some variations, may be part of a sealing face layer. This support layer 3225 is typically an air-permeable layer that helps support the flexible layer. In the variation shown in FIGS. 32A-36, the mesh or support layer 3225 is not necessary, but may help maintain the stiffness of the billowing airflow resistor. In FIG. 32C, the different layers may be adhesively secured together. For example, two adhesive rings 3227, 3227′ are shown helping secure the flexible layer and the sealing face layer to each other and to the rest of the nasal device, including the holdfast. These adhesive rings may be formed of double-sided adhesive. In some variations the layers are secured to each other and/or to the device by additional or optional methods. For example, the layers may be secured together using mechanical fasteners, clips, clamps, staples, etc. In some variations the sealing face layer 3223 in FIG. 32C is adhesively secured (e.g., glued) to the mesh/support layer 3225.

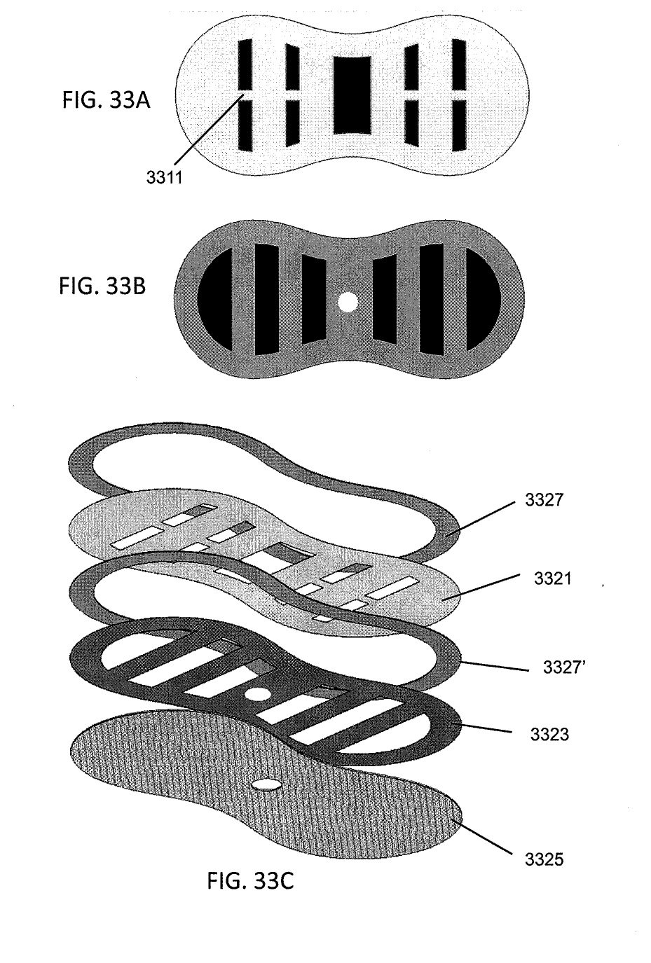

FIGS. 33A-33C illustrate another variation of a billowing airflow resistor that is very similar to the variation shown in FIG. 32A-32C, except that the strips in the flexible layer 3321 are connected 3311. Connecting the strips in this manner may limit the motion of the flexible layer during inhalation, e.g., limiting how much the layer will separate from the sealing face layer. These connections effectively reduce the size of the openings in the flexible layer. This may also reduce noise by reducing the length the unsecured edges of the flexible layer. Additionally, these connections may prevent the flexible layer from pushing through the openings in the sealing face layer during exhalation.

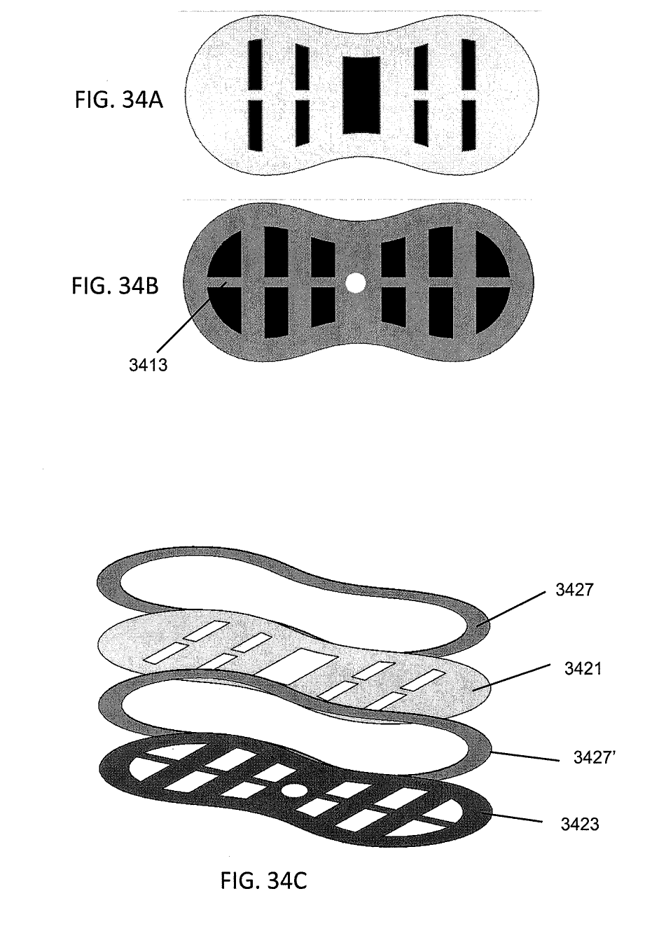

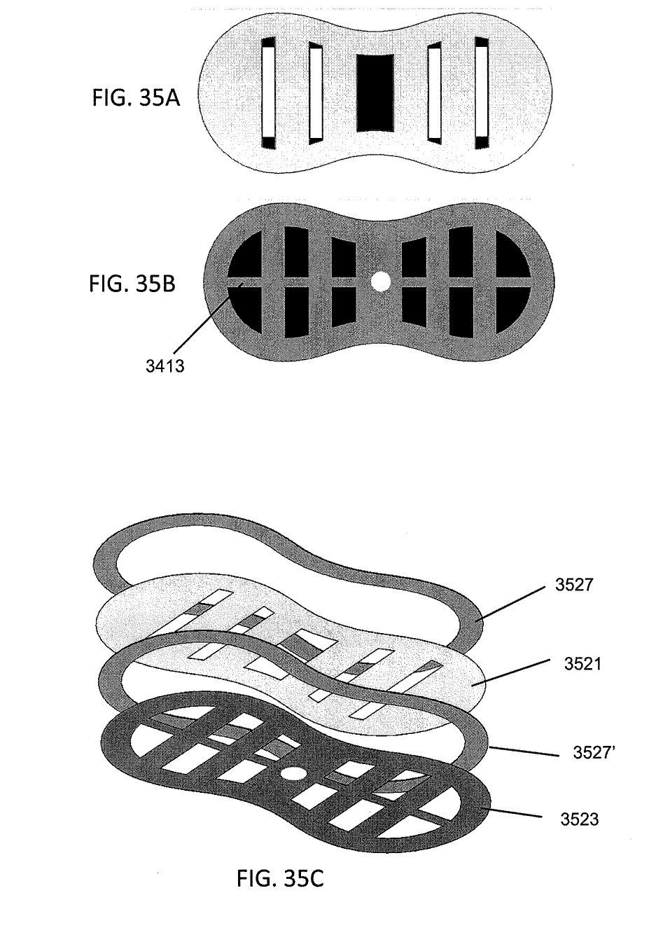

FIGS. 34A-34C and FIGS. 35A-35C show similar variations in which the sealing face layer (in FIG. 34B) also includes additional cross-supports 3413, 3513. These additional cross-supports provide additional surface against which the flexible layer can seal. These cross-supports or connections may also help prevent the flexible layer from pushing through the openings in the sealing face layer during exhalation. The cross-supports 3413, 3513 extend across the openings, as shown. In some variations, the openings may instead be mesh or otherwise air-permeable regions of the sealing face layer. FIGS. 34C and 35C also illustrate exploded views of billowing airflow resistors including the sealing face layer shown in FIGS. 34B and 35B. The optional support (mesh) layer shown in FIGS. 32C and 33C is not included in FIGS. 34C and 35C.

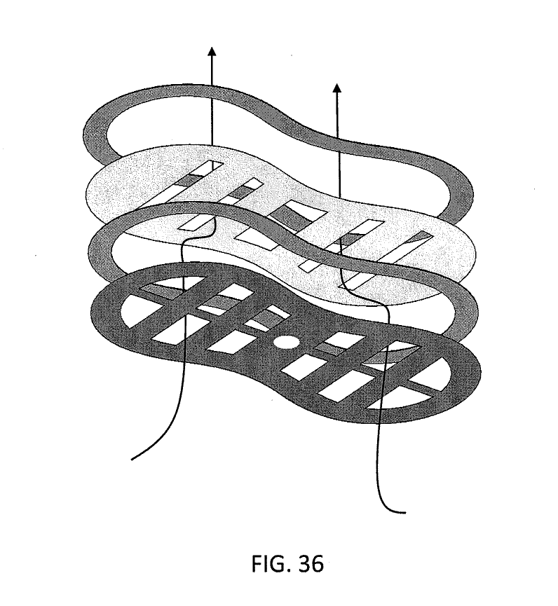

FIG. 36 illustrates the flow of air through the billowing airflow resistor variation shown in FIGS. 35A-35C during inhalation. In this example, during exhalation the flexible layer is pushed against the sealing face layer, and air may pass through the device through the leak pathway, such as the center hole. During inhalation, the strips forming the flexible layer separate from the sealing face layer, although their ends may be constrained. Thus, the flexible layer billows up from the sealing face layer. Separation of the flexible layer from the sealing face layer allows air to flow through the billowing airflow resistor, as indicated by the arrows in the exploded view of FIG. 36.

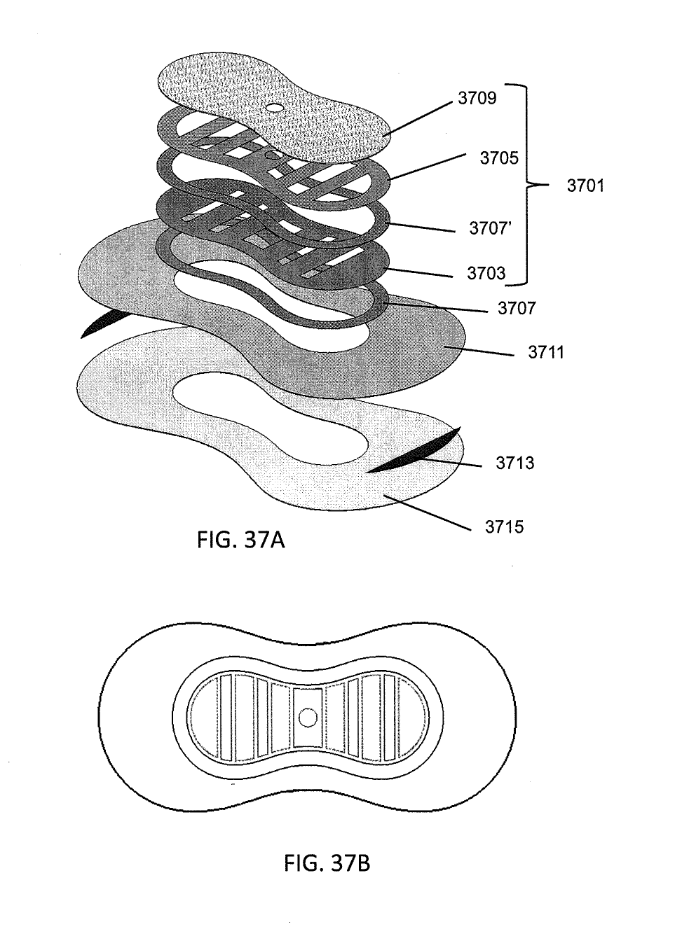

FIG. 37A shows an exploded view of a nasal device including a billowing airflow resistor. A top view of an assembled device is shown in FIG. 37B (some of the layers have been made transparent to better illustrate the assembly in FIG. 37B). In FIG. 37A, the billowing airflow resistor 3701 includes a flexible layer 3703, a sealing face layer 3705, and an additional support layer (mesh) 3709. These layers are secured together and to an adhesive holdfast layer 3711 using a perimeter (e.g., ring) of double-sided adhesive 3707, 3707′. The adhesive holdfast layer 3711 may also include a protective backing 3715, and non-stick regions that may be formed by adhesively securing paper or other non-tack regions 3713 to the edges of the adhesive holdfast 3711. The non-stick regions may be helpful for grasping and applying the device.

In operation, a nasal device having a billowing airflow resistor, such as the one shown in FIG. 37B, may be applied by a subject so that it covers the subject's nostrils, placing the billowing airflow resistor in communication with the nasal cavity. The exemplary device shown in FIG. 37B is a whole-nose device. The subject may be instructed on how to apply the device. Prior to applying the device the subject may be instructed to clean the region of the face that the device will be applied (e.g., around the nostrils) so that they are substantially clean and oil-free. The subject may then be instructed to remove the adhesive protective backing by unpeeling it, exposing the adhesive holdfast. Optionally, the subject may then be instructed to pull the skin around the nostrils taut. For example, the instructions may describe lowering the upper lip so that it extends downward as far as is comfortable (e.g., over the upper teeth), to provide better access to the region around the nostrils. The instructions may then describe placing the adhesive holdfast against the skin around the nostrils, and rubbing, pushing and/or brushing around the adhesive to assure that a seal with the skin and the adhesive holdfast is made. The instructions may also include steps for re-applying the device (or a fresh device) if the seal is not adequate. Leaks in the seal between the holdfast and the nose may be determined by feel or by the motion of air around the device, rather than through the airflow resistor.

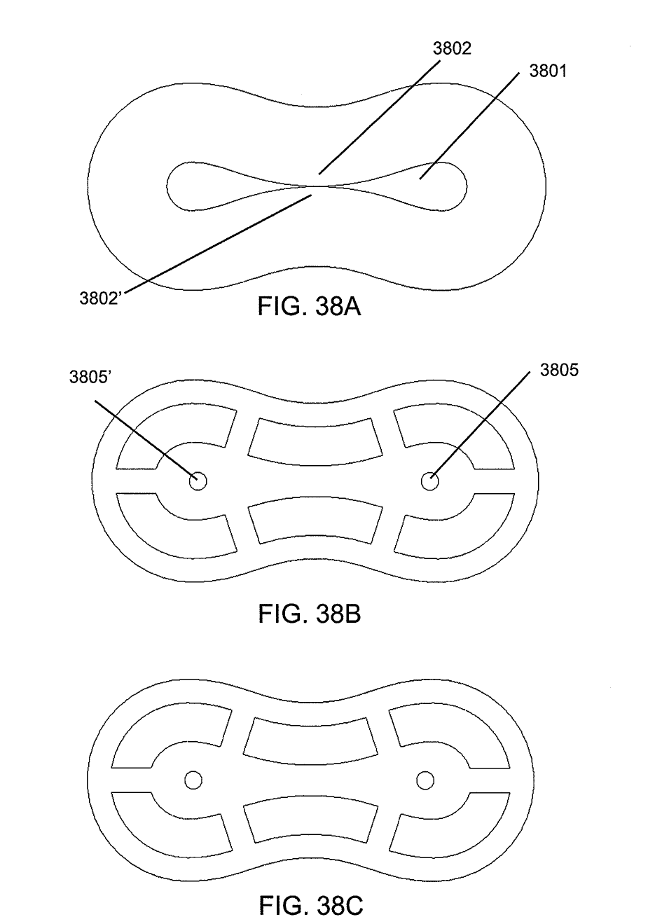

FIGS. 38A-38C show another variation of an airflow resistor that billows. This variation includes a flexible layer (shown in FIG. 38A) and a sealing face layer (shown in FIG. 38B). In this variation, the flexible layer is approximately “8” shaped, and includes a central cut-out region 3801. FIG. 38C illustrates the combined view of the flexible layer and the sealing face layer; the flexible layer has been made transparent to better see the relationship between the layers. In FIG. 38C, the overlap between the flexible layer and the sealing face layer allows the edges of the flexible layer to rest against the sealing face layer during exhalation. The sealing face layer also includes support regions (beams) that support the flexible layer during exhalation. Two leak pathways 3805, 3805′ through the sealing face layer and the flexible layer are also included.

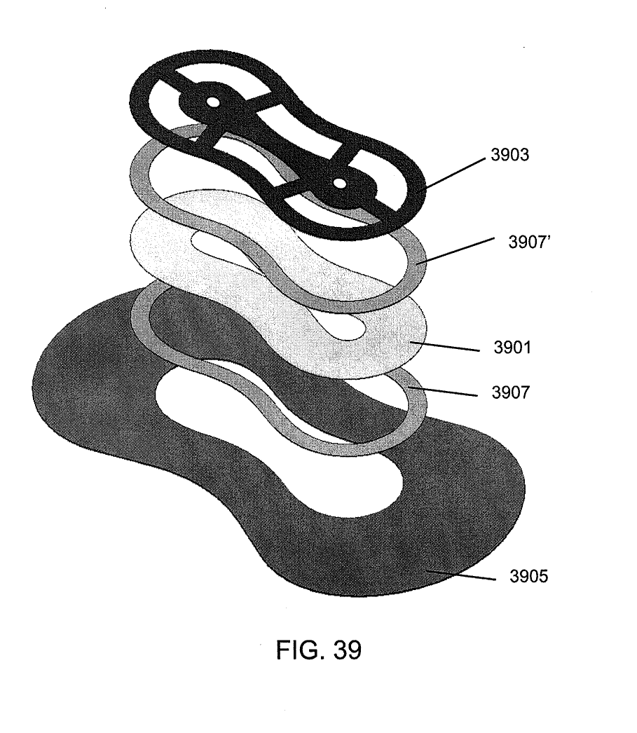

An exploded view of an adhesive nasal device including an airflow resistor such as the one shown in FIGS. 38A-38C is illustrated in FIG. 39. The sealing face layer 3903 is adhesively secured to the flexible layer 3901 and to the adhesive holdfast 3905 via double-sided adhesive rings 3907, 3907′. The billowing airflow resistor is secured in an opening through the adhesive holdfast in this example. The adhesive holdfast is arranged so that the flexible layer is closest to the subject nasal cavity when the device is worn (e.g. the adhesive side of the adhesive holdfast is the side that is not visible in FIG. 39).

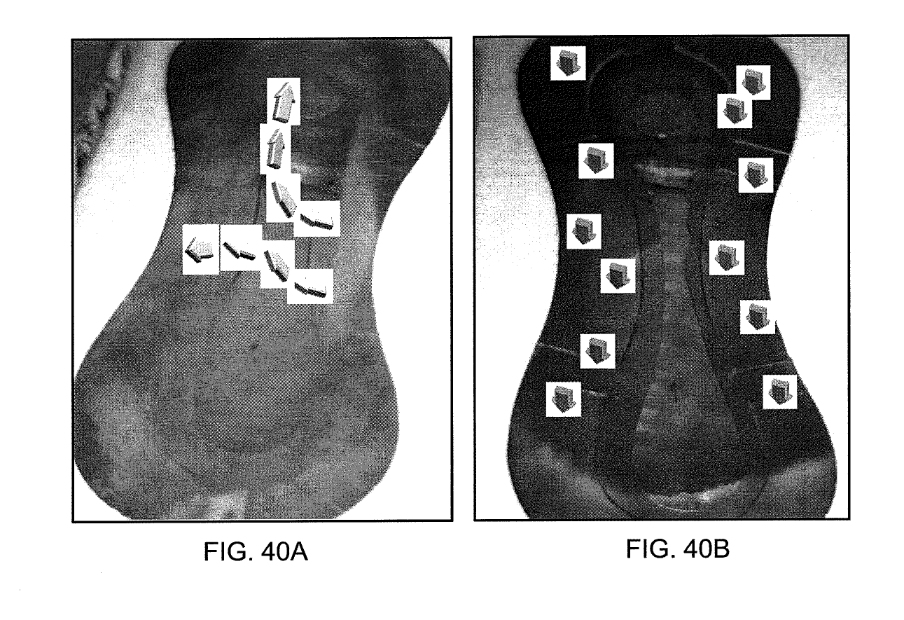

In use, the device shown in FIG. 39 is worn by a subject so that during inhalation, the flexible layer separates from the sealing face layer. In this variation, the flexible layer is drawn towards the nasal cavity by the negative pressure from the nasal cavity, and the central region of the flexible layer is separated from the sealing face layer. An example of this is shown in FIG. 40A. The arrows in FIG. 40A illustrate the entry of air under the flexible layer of the device as it is separated from the supporting sealing face layer. During exhalation, the force of the positive pressure applied from the nasal cavity pushes the flexible layer against the sealing face layer, closing the openings, and preventing air from flowing under and around the flexible layer; air passes only through the leak pathways. This is illustrated in FIG. 40B by the arrows.

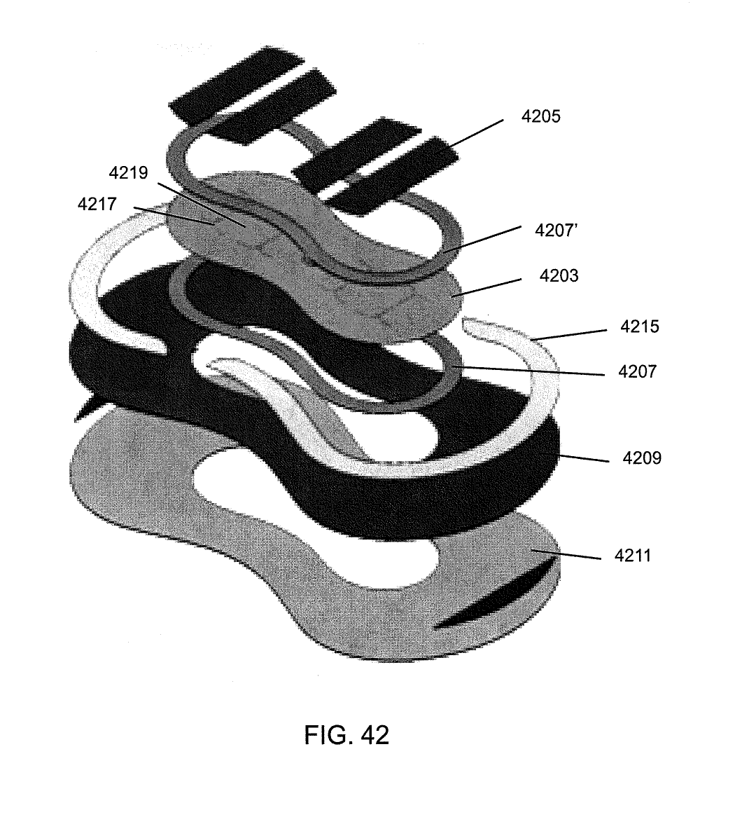

The billowing airflow resistors described herein are very similar, and share similar characteristics with, the flap valve airflow resistors previously described, and incorporated above by reference. As mentioned above, the billowing valves are typically not as loose (e.g., readily opened) as flap valves, since billowing valves may be secured so that the flexible layer is constrained along the edge, allowing it to separate from the support layer in parallel to the support layer. In some variations, an airflow resistor forming part of a nasal device may include features of both a flap and a billowing airflow resistor. For example, an airflow resistor may be a hybrid of a flap valve and a billowing valve. The airflow resistor shown in FIGS. 38A-40B is one such example; in this variation, the regions of the edge portion of the flexible layer near the center of the airflow resistor open almost flap-like. These portions of the flexible layer 3802 and 3802′ have a free edge that is curved outwardly, and thus may more freely ‘flap’ open and substantially leave the plane of the sealing face layer. In general, the more curved this free edge is, the more like a flap valve this portion of the airflow resistor will behave. The regions of the flexible layer that are not part of the outwardly curved (loose) edge behave more like billowing valves, since these regions will separate (billow) away from the sealing face layer while remaining substantially parallel to the sealing face layer. FIG. 42 shows an exploded view of another variation of a hybrid billowing and flap valve.

In FIG. 42, the hybrid flap/billowing airflow resistor includes a support layer 4205 and a flexible layer 4203, that may be adhesively connected by peripheral adhesive 4207′. In this example, the support layer 4205 includes strips of relatively stiff material against which openings or slits on the flexible layer 4203 may rest during exhalation, a mesh backing (not shown) may also be included). The flexible layer 4203 includes a plurality of cuts or slits 4217 which have been made so that outwardly curving free ends are formed. These cuts in this example have an “H” shape. These free ends may flap open during inhalation, and the region surrounding the inwardly curving free ends (e.g., the region between adjacent cuts 4209) may billow up and away from the support layer 4205. In the nasal device variation shown in FIG. 42, the support layer is formed by strips of material and a strip of mesh material (not shown). The mesh backing region may underlie the ‘flap’ regions of the flexible layer. In FIG. 42, the hybrid airflow resistor may be adhesively secured 4207 to an adhesive holdfast 4209 having a backing material 4211 and a support frame 4215.

As mentioned above, the nasal devices described herein may include any appropriate holdfast, including adhesive holdfasts for securing the device in communication with a nasal cavity. An adhesive holdfast may include one or more adhesive surfaces that are suitable for use against a subject's body (e.g., skin and/or nasal cavity). Thus, the adhesive holdfast may include a biocompatible adhesive. The adhesive holdfast may facilitate the positioning and securing of the device in a desired location with respect to the subject's nose, such as over, partially over, partially within, or within (e.g., substantially within) a nostril. An adhesive holdfast may be configured to secure the device to any appropriate region of the subject's nose, nasal passage, or nasal cavity, including the nostrils, nares or nasal chambers, limen, vestibule, greater alar cartilage, alar fibrofatty tissue, lateral nasal cartilage, agger nasi, floor of the nasal cavity, turbinates, sinuses (frontal, ethmoid, sphenoid, and maxillary), and nasal septum. The term “nasal cavity” may refer to any sub-region of the Nasal Fossa (e.g., a single nostril, nare, or nasal chamber) and includes or is defined by any of the anatomical terms listed above.

In general, an adhesive holdfast is configured to be applied predominantly to the outside of the nose (e.g., the skin surrounding the nasal opening or nostril). In some versions, the holdfast may also form a seal between the respiratory device and the nose, so that all or most of the air exchanged between the outside of the patient and the nostril must pass through the respiratory device. In some versions, the holdfast seals the device in communication with the nose completely, so that all air through the nostril (or nostrils) must be exchanged through the device. In some versions, the holdfast seal is incomplete, so that only some of the air exchanged between the patient and the external environment passes through the device. As used herein, “air” may be air from environment external to the patient, or it may be any respiratory gas (e.g., pure or mixed oxygen, CO2, heliox, or other gas mixtures provided to the user).

The adhesive holdfast may be flexible so that it conforms to the surface of the subject's skin, which may be relatively irregularly shaped, and may include hair and the like. In some variations, the adhesive holdfast is made of a material that permits the passage of water vapor, liquid water, sweat and/or oil, which may enhance comfort. The adhesive holdfast may also include a texture or patterned relief surface to enhance bonding to the subject's nose region.

The adhesive holdfast may be made of layers. Thus, the adhesive holdfast may be referred to as a layered holdfast (or layered adhesive holdfast) For example, the adhesive holdfast may include a substrate layer to which a biocompatible adhesive is applied. The substrate is typically a flat (predominantly 2-sided) material that is flexible. An adhesive may be present on at least one surface of the substrate, allowing it to adhere to the subject's nasal region. In some variations, the substrate layer is itself adhesive without needing an additional adhesive. An additional protective cover may also be removably attached to the adhesive of the adhesive layer. The protective cover may allow the device (and particularly the adhesive holdfast) to be manipulated without inadvertently sticking the device to the fingers or other parts of the body and it may also prevent contamination of the adhesive. The liner may be a removable paper or other film that can be peeled off or otherwise removed to expose the adhesive. In some variations, the adhesive of the adhesive holdfast is activatable. For example, the adhesive becomes ‘sticky’ only after exposure to an activator (e.g., water, air, light, etc.). In some variations, an adhesive could be applied to the nose or the holdfast layer in a liquid form first, and then the device applied.

In some variations, a protective cover is not used. As already mentioned, in some variations, the substrate and adhesive are a single layer, so that the substrate comprises an adhesive material, or a material that can be activated to become adhesive. The adhesive holdfast may comprise any appropriate material. For example, the adhesive substrate may be a biocompatible material such as silicone, polyethylene, or polyethylene foam. Other appropriate biocompatible materials may include some of the materials previously described, such as biocompatible polymers and/or elastomers. Suitable biocompatible polymers may include materials such as: a homopolymer and copolymers of vinyl acetate (such as ethylene vinyl acetate copolymer and polyvinylchloride copolymers), a homopolymer and copolymers of acrylates (such as polypropylene, polymethylmethacrylate, polyethylmethacrylate, polymethacrylate, ethylene glycol dimethacrylate, ethylene dimethacrylate and hydroxymethyl methacrylate, and the like), polyvinylpyrrolidone, 2-pyrrolidone, polyacrylonitrile butadiene, polyamides, fluoropolymers (such as polytetrafluoroethylene and polyvinyl fluoride), a homopolymer and copolymers of styrene acrylonitrile, cellulose acetate, a homopolymer and copolymers of acrylonitrile butadiene styrene, polymethylpentene, polysulfones polyimides, polyisobutylene, polymethylstyrene and other similar compounds known to those skilled in the art. Structurally, the substrate may be a film, foil, woven, non-woven, foam, or tissue material (e.g., poluelofin non-woven materials, polyurethane woven materials, polyethylene foams, polyurethane foams, polyurethane film, etc.).

In variations in which an adhesive is applied to the substrate, the adhesive may comprise a medical grade adhesive such as a hydrocolloid or an acrylic. Medical grade adhesives may include foamed adhesives, acrylic co-polymer adhesives, porous acrylics, synthetic rubber-based adhesives, silicone adhesive formulations (e.g., silicone gel adhesive), and absorbent hydrocolloids and hydrogels. In some variations the substrate itself is an adhesive. For example, the substrate may be a hydrocolloid.

In some variations, the adhesive is a structural adhesive. For example, the adhesive may adhere based on van der Walls forces. Patents no. U.S. Pat. No. 7,011,723, U.S. Pat. No. 6,872,439, U.S. Pat. No. 6,737,160, and U.S. Pat. No. 7,175,723 describe setal-like structures whose shape and dimension provide adhesive force. These patents are herein incorporated by reference in their entirety.

The removable liner layer may be made of any appropriate matter that may be released from the adhesive. For example, the liner material may comprise craft paper. In some variations, the liner material comprises polyethylene film, or polyethylene coated paper (e.g. kraft paper). The liner may be any of the other materials described herein.

In general, any of the materials commonly used in the manufacture of bandages (particularly disposable bandages such as Band-Aids™), ostomy kits, and wound care products may be used in any or all components of devices described herein. An adhesive layer (or an adhesive holdfast layer) may be formed in any appropriate method, particularly those described herein. For example, an adhesive layer may be formed by cutting (stamping, die cutting, laser cutting, etc.) the adhesive substrate, biocompatible adhesive, and protective cover into the desired shape. Multiple steps may be used to form the adhesive layer. For example, the adhesive layer may be formed by cutting (or otherwise forming) the outer perimeter, then by cutting (or otherwise forming) an inner opening.

In general, the adhesive holdfast may comprise any appropriate shape that allows the airflow resistor to be positioned with respect to one or both nasal passages so that some (or most) of the airflow through the nasal passages must pass through the adhesive nasal device.

It is not necessary that the entire adhesive holdfast region include an adhesive, although many of the substantially flat holdfast regions described in the preceding figures may have a biocompatible adhesive over much of the skin-contacting surface (although it may be covered by a protective cover that can be at least partially removed later). In some variations only a subset of the holdfast region (including the outer layer) includes an adhesive. For example the region adjacent to the rim body may not include an adhesive, or the region beneath the tabs or grips may not include an adhesive.

In some variations, the adhesive nasal devices described herein are adapted to fit different users having a diversity of sizes and shapes, particularly the shapes and sizes of their noses. As already described, the devices, including particularly the adhesive holdfast region, may be configured so that it is adaptable to different nose shapes. In some variations, the holdfast region may extend into the nostril, rather than just adhering around the outer surface of the nasal passages. For example, the adhesive holdfast may include a region that projects into the nostril, and can be secured against the walls of the nostril. In some variations, the internally-projecting regions may comprise a compressible material (e.g., a foam or the like) so that they may be secured within the nasal passages, and/or may cushion the inner rim base region (or any other portion of the adhesive nasal device) that projects into the subject's nostrils. Thus, in some variations, the inwardly-projecting portion of the holdfast is smaller than the nasal opening, and does not necessarily contact the sides of the subject's nasal passage.

In some embodiments, one or more components of the device are impregnated with, contain, or are coated with one or more compounds that may be inhaled during use. The presence of airflow, heat or other conditions may facilitate the release of the compound into the inhaled air or surrounding tissues. The compound may be herbal (such as menthol or lavender), chemical or pharmaceutical (such as an antihistamine or anti asthma drug) in nature. Depending on the compound, the user might experience a pleasant aroma (which may soothe or promote sleep or activity) or medical benefits, such as nasal decongestion or asthma relief. The compound may be inhaled during all or at least a portion of the time the user is wearing the device. The compounds may be used as part of treatment of a sleep apnea, snoring, or may find use in other embodiments for other medical conditions.

In still other embodiments, the device may include a filter that removes particulate matter from external air upon inhalation. Particulate matter that would be removed may include dust and allergens. This invention may be embodied within a sleep apnea device, snoring device, a respiratory device, or comprise a stand-alone device.

Other materials of interest include any materials that can serve as filters for allergens, pollen, dander, smog, etc. By providing a filter within the device, sinusitis, sleep apnea, snoring, hay fever, allergic rhinitis, and other allergic respiratory conditions may be reduced or prevented. This filter may in fact be part of the airflow resistor (e.g., a support layer) or may be a separate component of the device. Any suitable filtering material known to those skilled in the art may be used with the respiratory devices described herein. Such materials include, but are not limited to, activated carbon charcoal filters, hollow-fiber filters, and the like.

In some versions, the respiratory device may comprise a filter that remains in the path of inhalation and/or exhalation during use. In some versions, the filter material remains in the path of both inspiratory and expiratory airflow. This filter material may not appreciably alter resistance to airflow in either direction, or it may alter airflow to substantially the same degree in both directions (inhalation and exhalation). In some versions, the filter comprises a material having a large pore size so that airflow is not significantly inhibited.

In some versions, the device is used with an active agent. In some versions, the active agent comprises a drug. An active agent (e.g., a medicament) or other compound can be placed in or on the device to deliver the active agent into the mouth, tongue, hard and soft palates, sinuses, nose, nasal cavity, pharynx, vocal cords, larynx, airways, lungs, trachea, bronchi, bronchioles, alveoli, air sacs, or any tissues that are exposed to inspiratory or expiratory airflow. In some cases, the active agent may be embedded or impregnated in the device or components of the device. In some cases the active agent is a coating. An active agent may comprise any compound that is in some way useful or desirable for the patient. For example, the active agent may be any odorant, including: menthol, phenol, eucalyptus, or any agent that provides a fragrance in the inspired air. Alternatively, an active agent may comprise a drug with beneficial effects, such as beneficial vasculature effects. For example, an active agent may comprise a drug that affects the blood vessels (oxymetazoline or any other vasoactive compound), nasopharynx, airways or lungs (albuterol, steroids, or other bronchoconstriction or bronchodilation compounds). An active agent may comprise, for example, an antibiotic or a steroid. The above list of active agents is not meant to be limiting.

An active agent may be placed in or on any portion of the device. Furthermore, the location of the active agent within the respiratory device may specifically guide the delivery of the active agent. For example, in versions of the respiratory device configured to be placed inside a respiratory cavity, when the holdfast comprises an active agent (e.g., coated, embedded or otherwise part of the holdfast), the drug may be delivered through the mucus membranes of the respiratory cavity. In another example, an active agent may be included as a powder or releasable coating that may be aerosolized and delivered within the respiratory system. Thus, an active agent may be on a surface of the device (e.g., the passageway, holdfast or airflow resistor) or embedded within any surface of the device. A separate drug-containing region may also be included in the device. The addition of an active agent may be of particular interest in treating allergies and sinusitis. Respiratory devices (with or without airflow resistors) may therefore comprise active agents such as menthol or other fragrant compounds.

In some variations of the device an aligner is used to help align the device (e.g., the airflow resistor) with one or both of a subject's nostrils. An aligner may include a tactile aligner that may be felt by the subject's nose or hands, a visual aligner (e.g., a color, pattern, or other marking) that may be seen by the subject, or a structural aligner that inserts at least partly in or around the subject's nostril(s), or any combination of these. In some variations, an aligner may also help maintain the alignment of the device with the subject's nostril(s), for example, by helping maintain the patency of the nostril opening.

While the devices, systems, and methods for using them have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A nasal device configured to inhibit nasal exhalation more than nasal inhalation, the device comprising:
a holdfast configured to secure the device in communication with the subject's nasal cavity;
an opening; and
a billowing airflow resistor extending across the opening, the billowing airflow resistor comprising:
a first layer, wherein the first layer is flexible, and
a second layer adjacent to the first layer, wherein the first layer is configured to lie against the second layer during exhalation through the opening, and further wherein the first layer is configured to separate from the second layer while remaining substantially parallel to the second layer during inhalation through the opening.

2. The device of claim 1, further comprising a leak pathway through the device configured to be opening during both inhalation and exhalation.

3. The device of claim 1, wherein the billowing airflow resistor further comprises a support layer extending across the opening and adjacent to the second layer.

4. The device of claim 3, wherein the support layer comprises a mesh.

5. The device of claim 1, wherein the opening is at least partially surrounded by the holdfast.

6. The device of claim 1, wherein the holdfast comprises an adhesive layer configured to adhesively secure the device to the subject.

7. The device of claim 1, wherein the holdfast comprises a flexible adhesive holdfast layer that extends in substantially the same plane as the billowing airflow resistor.

8. The device of claim 1, wherein the first layer of the billowing airflow resistor comprises a plurality of strips extending across the opening.

9. The device of claim 1, wherein the first layer is secured across the opening in at least two regions.

10. The device of claim 1, wherein the first layer includes a plurality of openings there through.

11. The device of claim 1, wherein the second layer comprises a relatively stiff material.

12. The device of claim 1, wherein the second layer comprises a mesh.

13. The device of claim 1, wherein the first layer comprises one or more opening that are offset from openings in the second layer when the first and second layer lie against each other during exhalation.

14. The device of claim 1, wherein the billowing airflow resistor is configured so that during exhalation through the opening, the resistance through the device is between about 0.001 and about 0.25 cm H₂O/(ml/sec), and during inhalation through the opening, the resistance through the device is between about 0.0001 and about 0.02 cm H2O/(ml/sec).

15. A nasal device configured to inhibit nasal exhalation more than nasal inhalation, the device comprising:
a holdfast configured to secure the device in communication with the subject's nasal cavity;
an opening; and
a billowing airflow resistor extending across the opening, the billowing airflow resistor comprising:
a flexible layer secured across the opening, and
a sealing face layer adjacent to the flexible layer,
wherein the billowing airflow resistor is configured so that during exhalation through the opening, the flexible layer presses against the sealing face layer; and during inhalation through the opening the flexible layer separates slightly from the sealing face layer while remaining substantially parallel to the sealing face layer.

16. The device of claim 15, further comprising a leak pathway through the device configured to be opening during both inhalation and exhalation.

17. The device of claim 15, wherein the holdfast comprises an adhesive layer configured to adhesively secure the device to the subject.

18. The device of claim 15, wherein the opening is at least partially surrounded by the holdfast.

19. The device of claim 15, wherein the holdfast comprises a flexible adhesive holdfast layer that extends in substantially the same plane as the billowing airflow resistor.

20. The device of claim 15, wherein the flexible layer of the billowing airflow resistor comprises a plurality of strips extending across the opening.

21. The device of claim 15, wherein the flexible layer is secured across the opening in at least two regions.

22. The device of claim 15, wherein the flexible layer includes a plurality of openings there through.

23. The device of claim 15, wherein the sealing face layer comprises a relatively stiff material.

24. The device of claim 15, wherein the flexible layer comprises one or more opening that are offset from openings in the sealing face layer when the flexible layer and the sealing face layer press against each other during exhalation.

25. The device of claim 15, wherein the billowing airflow resistor is configured so that during exhalation, the resistance through the device is between about 0.001 and about 0.25 cm H₂O/(ml/sec), and during inhalation through the opening, the resistance through the device is between about 0.0001 and about 0.02 cm H2O/(ml/sec).

26. The device of claim 15, wherein the sealing face layer comprises a mesh.

27. The device of claim 15, further comprising a mesh layer extending across the opening, on the other side of the sealing face layer away from the flexible layer.

28. A nasal device configured to inhibit nasal exhalation more than nasal inhalation, the device comprising:
a holdfast configured to secure the device in communication with the subject's nasal cavity;
an opening at least partially surrounded by the holdfast; and
a billowing airflow resistor extending across the opening, the billowing airflow resistor comprising:
a first layer, and
a second layer adjacent to the first layer,
wherein the first layer is configured to lie against the second layer during exhalation through the opening to provide a resistance of between about 0.001 and about 0.25 cm H₂O/(ml/sec) through the device, and
further wherein the first layer is configured to separate from the second layer during inhalation through the opening, while remaining substantially parallel to the second layer to provide a resistance of between about 0.0001 and about 0.02 cm H2O/(ml/sec) through the device.

29. A method of treating a respiratory disorder, the method comprising:
securing a nasal device in communication with the subject's nasal cavity, wherein the nasal device comprises a billowing airflow resistor having a flexible layer and a sealing face layer;
driving the flexible layer against the sealing face layer during exhalation to create a resistance to exhalation through the nasal device; and
separating the flexible layer from the sealing face layer during inhalation while maintaining the layers substantially in parallel to create a resistance to inhalation through the nasal device that is less than the resistance to exhalation.

30. The method of claim 29, wherein the resistance to exhalation is between about 0.001 and about 0.25 cm H₂O/(ml/sec).

31. The method of claim 29, wherein the resistance to inhalation is between about 0.0001 and about 0.02 cm H2O/(ml/sec).

32. The method of claim 29, wherein the step of securing the nasal device comprises adhesively securing the device over both of a subject's nostrils.

33. The method of claim 29, wherein the step of securing the nasal device comprises securing the nasal device at least partially within a subject's nostril.

34. The method of claim 29, wherein the respiratory disorder treated comprises a sleeping disorder selected from the group consisting of: snoring and sleep apnea.

35. A nasal respiratory device adapted for measuring intranasal pressure while secured in communication with a subject's nasal cavity, the device comprising:
a device body having an opening configured to be placed in communication with the nasal cavity, wherein the device body includes an inner side configured to face the nasal cavity and an outer side configured to face away from the nasal cavity when the device is worn;
an airflow resistor in communication with the opening, wherein the airflow resistor is configured to increase the resistance to air exhaled through the opening of the nasal device more than the resistance to air inhaled through the opening; and
a pressure sensor exposed to the inner side of the device and configured to sense intranasal pressure when the device is worn.

36. The device of claim 35, wherein the pressure sensor comprises a cannula having a distal opening configured to communicate with the intranasal region when the device is worn by a subject.

37. The device of claim 35, wherein the pressure sensor is not exposed directly to flow through the nasal respiratory device.

38. The device of claim 35, wherein the pressure sensor comprises a pressure transducer.

39. The device of claim 35, wherein the airflow resistor comprises a flap valve.

40. The device of claim 35, further comprising an adhesive holdfast.

41. A nasal respiratory device adapted for measuring intranasal pressure while secured in communication with a subject's nasal cavity, the device comprising:
a device body having an opening configured to be placed in communication with the nasal cavity;
an airflow resistor in communication with the opening, wherein the airflow resistor is configured to increase the resistance to air exhaled through the opening more than the resistance to air inhaled through the opening; and
a cannula passing through the nasal device having a distal opening configured to be exposed to intranasal pressure when the device is worn, wherein the cannula is configured to connect to a pressure transducer for sensing intranasal pressure.

42. The device of claim 41, wherein the airflow resistor comprises a flap valve.

43. The device of claim 41, wherein the distal opening of the cannula is protected from a flow pathway through the device.

44. The device of claim 43, wherein the distal opening of the cannula is a side opening.

45. The device of claim 41, wherein the cannula passes through the airflow resistor.

46. The device of claim 41, wherein the cannula secures the airflow resistor within the opening through the nasal device.

47. The device of claim 41, further comprising an adhesive holdfast.

48. A system for monitoring intranasal pressure, comprising:
a passive-resistance nasal respiratory device including an airflow resistor configured to inhibit exhalation more than inhalation;
an intranasal cannula configured to pass through the nasal respiratory device; and
connector tubing configured to connect the intranasal cannula to a pressure sensor.

49. The system of claim 48, further comprising a pressure sensor configured to connect to the intranasal cannula via the connector tubing.

50. The system of claim 48, further comprising a filter configured to be placed in communication with the intranasal cannula.

51. The system of claim 48, wherein the passive-resistance nasal respiratory device comprises an adhesive holdfast.

52. A method of monitoring treatment of a sleep disorder comprising:
securing a nasal respiratory device in communication with the subject's nasal cavity without covering the subject's mouth, wherein the respiratory device includes a passive airflow resistor configured to inhibit exhalation more than inhalation and an intranasal cannula passing through the nasal respiratory device, so that the intranasal cannula communicates with the patient's intranasal space; and
monitoring intranasal pressure using the cannula.

53. An adapter device for use with a disposable nasal device, the adapter device comprising:
a housing configured to be secured to a subject's nose without covering the subject's mouth;
an opening through the housing, the opening having a perimeter region that is configured to secure a passive nasal device thereto so that the passive nasal device may inhibit exhalation more than inhalation through the subject's nose when the passive nasal device is secured to the perimeter region; and
a holdfast configured to seal the housing to the subject's nose so that substantially all of the airflow to and from the subject's nose passes through the disposable passive nasal device secured to the perimeter region.

54. The device of claim 53, wherein the housing comprises a nose mask.

55. The device of claim 53, wherein the housing comprises a sealing edge region configured to secure the edge of the housing to a subject's face.

56. The device of claim 53, wherein the opening through the housing is located opposite to a subject's nostril or nostrils when the adapter device is worn by the subject.

57. The device of claim 53, wherein the holdfast comprises a strap.

58. The device of claim 53, wherein the holdfast comprises an adhesive.

59. The device of claim 53, wherein the holdfast comprises a compressible sealing ring around the perimeter of the housing.

60. The device of claim 53, further comprising a passive nasal device configured to inhibit exhalation more than inhalation.

61. A method of applying a disposable passive nasal device to an adapter nose mask, wherein the passive nasal device is configured to inhibit exhalation more than inhalation, the method comprising:
exposing a perimeter region around an opening through an adapter nose mask, wherein the adapter nose mask further comprises a holdfast for sealing the adapter nose mask over or in the subject's nose without substantially covering the subject's mouth; and
securing the holdfast of the passive nasal device over or across the opening through the adapter nose mask so that when the adapter nose mask is worn by a subject, substantially all of the air flowing into and out of the subject's nose passes through the passive nasal device, to inhibit exhalation more than inhalation.

62. The method of claim 61, wherein the step of securing the holdfast of the passive nasal device over or across the opening through the adapter nose mask comprises adhesively securing the passive nasal device to the adapter nose mask.

63. The method of claim 61, wherein the step of securing the holdfast of the passive nasal device over or across the opening through the adapter nose mask comprises expanding a compressible holdfast of the passive nasal device at least partially within the opening through the adapter nose mask.

64. A method of applying an adapter to a passive nasal device so that the passive nasal device may be re-used, wherein the passive nasal device comprises an airflow resistor configured to inhibit exhalation more than inhalation and an adhesive holdfast, the method comprising applying a replacement adhesive holdfast over the adhesive holdfast of the passive nasal device.
